SEQUENCE LISTING (22 sequences)

SEQ ID NO: 1 - protein, 195 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTR

SEQ ID NO: 2 - protein, 124 aa, Homo sapiens
TVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARV

SEQ ID NO: 3 - protein, 169 aa, Homo sapiens
CYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLA

SEQ ID NO: 4 - protein, 142 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 5 - protein, 107 aa, Mus musculus
DTVMTQSHKIMSTSVGDRVSITCKASQDVSIGVAWYQQRPGQSPKLLIYSASYRYTGVPDRFTGSGSGTDFTFTISSVQAEDLAVYYCQQYYIYPYTFGGGTKLEIK

SEQ ID NO: 6 - protein, 119 aa, Mus musculus
EVQLQQSGPELVKPGTSVKISCKASGFTFTDYTMDWVKQSHGKSLEWIGDVNPNSGGSIYNQRFKGKASLTVDRSSRIVYMELRSLTFEDTAVYYCARNLGPSFYFDYWGQGTTLTVSS

SEQ ID NO: 7 - protein, 107 aa, Artificial Sequence [sequence omitted]

SEQ ID NO: 8 - protein, 119 aa, Artificial Sequence [sequence omitted]

SEQ ID NO: 9 - protein, 107 aa, Artificial Sequence
  Description of Artificial Sequence Synthetic polypeptide
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIK

SEQ ID NO: 10 - protein, 119 aa, Artificial Sequence
  Description of Artificial Sequence Synthetic polypeptide
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 11 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 217 aa, Artificial Sequence
  Description of Artificial Sequence Synthetic polypeptide
VHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 10 aa, Artificial Sequence
  Description of Artificial Sequence Synthetic peptide
  MOD_RES (10)..(10): Asp or Ser
GFTFTDYTMX

SEQ ID NO: 18 - protein, 17 aa, Artificial Sequence
  Description of Artificial Sequence Synthetic peptide
DVNPNSGGSIYNQRFKG

SEQ ID NO: 19 - protein, 10 aa, Artificial Sequence
  Description of Artificial Sequence Synthetic peptide
NLGPSFYFDY

SEQ ID NO: 20 - protein, 11 aa, Artificial Sequence
  Description of Artificial Sequence Synthetic peptide
KASQDVSIGVA

SEQ ID NO: 21 - protein, 7 aa, Artificial Sequence
  Description of Artificial Sequence Synthetic peptide
  MOD_RES (5)..(5): Arg or Leu
  MOD_RES (6)..(6): Tyr or Glu
  MOD_RES (7)..(7): Thr or Ser
SASYXXX

SEQ ID NO: 22 - protein, 9 aa, Artificial Sequence
  Description of Artificial Sequence Synthetic peptide
QQYYIYPYT

(19) United States
(12) Patent Application Publication
(10) Pub. No.: US 2020/0237910 A1
(43) Pub. Date: Jul. 30, 2020

(54) TREATMENT OF ADVANCED HER2 EXPRESSING CANCER

(71) Applicant: GENENTECH, INC., South San Francisco, CA (US)

(72) Inventors: Mary Beattie, San Francisco, CA (US); Melissa Brammer, Sedona, AZ (US)

(73) Assignee: GENENTECH, INC., South San Francisco, CA (US)

(21) Appl. No.: 16/828,045

(22) Filed: Mar. 24, 2020

Related U.S. Application Data
(63) Continuation of application No. 15/842,726, filed on Dec. 14, 2017.
(60) Provisional application No. 62/439,815, filed on Dec. 28, 2016.
(60) Provisional application No. 62/457,672, filed on Feb. 10, 2017.
(60) Provisional application No. 62/519,599, filed on Jun. 14, 2017.

(51) Int. Cl. A61K 39/395; A61P 35/04; C07K 16/32

(52) U.S. Cl. CPC A61K 39/39558; A61P 35/04; A61K 2039/55; A61K 2300/00; C07K 16/32

(57) ABSTRACT

Methods for the treatment of patients with HER2-positive, HER2-amplified and/or HER2-mutated advanced cancer by administration of pertuzumab plus trastuzumab are disclosed. In one aspect, the cancer is advanced HER2-positive, HER2-amplified and/or HER2-mutated colorectal, biliary, bladder, urothelial, salivary, lung, pancreatic, ovarian, prostate, or skin cancer. In another aspect, the cancer is HER2-positive, HER2-amplified and/or HER2-mutated colorectal, biliary, bladder, urothelial, salivary, lung, pancreatic, ovarian, prostate, or skin cancer that is refractory to one or more other treatment regimens.

### CROSS REFERENCE TO RELATED APPLICATIONS

This application is a continuation of U.S. application Ser. No. 15/842,726 filed on Dec. 14, 2017 which claims the benefit of priority under 35 USC § 119(e) of provisional Application No. 62/439,815, filed Dec. 28, 2016, provisional Application No. 62/457,672, filed Feb. 10, 2017 and provisional Application No. 62/519,599, filed Jun. 14, 2017, the full disclosures of which are hereby incorporated by reference in their entireties.

### SEQUENCE LISTING

The instant application contains a Sequence Listing which has been filed electronically in ASCII format and is hereby incorporated by reference in its entirety. Said ASCII copy, created on Mar. 18, 2020, is named P34056US5SEQLIST.txt and is 32,059 bytes in size.

### FIELD OF THE INVENTION

The present invention concerns the treatment of patients with HER2-positive, HER2-amplified and/or HER2-mutated cancer, such as colorectal, biliary, urothelial, bladder, salivary, lung, pancreatic, ovary, prostate, or skin (apocrine) cancer by administration of pertuzumab plus trastuzumab. In one aspect, the cancer is advanced HER2-positive, HER2-amplified and/or HER2-mutated colorectal, biliary, urothelial, bladder, salivary, lung cancer, pancreatic, ovary, prostate, or skin (apocrine). In another aspect, the cancer is HER2-positive, HER2-amplified and/or HER2-mutated colorectal, biliary, urothelial, bladder, salivary lung cancer, pancreatic, ovary, prostate, or skin (apocrine) that is refractory to one or more other treatment regimens.

### BACKGROUND OF THE INVENTION

Members of the HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2). Members of the receptor family have been implicated in various types of human malignancy.

A recombinant humanized version of the murine anti-HER2 antibody 4D5 (huMAb4D5-8, rhuMAb HER2, trastuzumab or HERCEPTN®; U.S. Pat. No. 5,821,337) is clinically active in patients with HER2-overexpressing metastatic breast cancers that have received extensive prior anti-cancer therapy (Baselga et al., J. Clin. Oncol. 14:737-744 (1996)).

Trastuzumab received marketing approval from the Food and Drug Administration Sep. 25, 1998 for the treatment of patients with metastatic breast cancer whose tumors overexpress the HER2 protein. At present, trastuzumab is approved for use as a single agent or in combination with chemotherapy or hormone therapy in the metastatic setting, and as single agent or in combination with chemotherapy as adjuvant treatment for patients with early-stage HER2-positive breast cancer. trastuzumab-based therapy is now the recommended treatment for patients with HER2-positive early-stage breast cancer who do not have contraindications for its use (Herceptin® prescribing information; NCCN Guidelines, version 2.2011). trastuzumab plus docetaxel (or paclitaxel) is a registered standard of care in the first-line metastatic breast cancer (MBC) treatment setting (Slamon et al. N Engl J Med. 2001; 344(11):783-792.; Marty et al. J Clin Oncol. 2005; 23(19):4265-4274).

Patients treated with the HER2 antibody trastuzumab are selected for therapy based on HER2 expression. See, for example, WO99/31140 (Paton et al.), US2003/0170234A1 (Hellmann, S.), and US2003/0147884 (Paton et al.); as well as WO01/89566, US2002/0064785, and US2003/0134344 (Mass et al.). See, also, U.S. Pat. Nos. 6,573,043, 6,905,830, and US2003/0152987, Cohen et al., concerning immunohistochemistry (IHC) and fluorescence in situ hybridization (FISH) for detecting HER2 overexpression and amplification. Thus, the optimal management of metastatic breast cancer now takes into account not only a patient's general condition, medical history, and receptor status, but also the HER2 status.

Pertuzumab (also known as recombinant humanized monoclonal antibody 2C4 (rhuMAb 2C4); Genentech, Inc, South San Francisco) represents the first in a new class of agents known as HER dimerization inhibitors (HDI) and functions to inhibit the ability of HER2 to form active heterodimers or homodimers with other HER receptors (such as EGFR/HER1, HER2, HER3 and HER4). See, for example, Harari and Yarden Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003).

Pertuzumab blockade of the formation of HER2-HER3 heterodimers in tumor cells has been demonstrated to inhibit critical cell signaling, which results in reduced tumor proliferation and survival (Agus et al. Cancer Cell 2:127-37 (2002)).

Pertuzumab has undergone testing as a single agent in the clinic with a phase Ia trial in patients with advanced cancers and phase II trials in patients with ovarian cancer and breast cancer as well as lung and prostate cancer. In a Phase I study, patients with incurable, locally advanced, recurrent or metastatic solid tumors that had progressed during or after standard therapy were treated with pertuzumab given intravenously every 3 weeks. Pertuzumab was generally well tolerated. Tumor regression was achieved in 3 of 20 patients evaluable for response. Two patients had confirmed partial responses. Stable disease lasting for more than 2.5 months was observed in 6 of 21 patients (Agus et al. Pro Am Soc Clin Oncol 22:192 (2003)). At doses of 2.0-15 mg/kg, the pharmacokinetics of pertuzumab was linear, and mean clearance ranged from 2.69 to 3.74 mL/day/kg and the mean terminal elimination half-life ranged from 15.3 to 27.6 days. Antibodies to pertuzumab were not detected (Allison et al. Pro Am Soc Clin Oncol 22:197 (2003)).

US 2006/0034842 describes methods for treating ErbB-expressing cancer with anti-ErbB2 antibody combinations. US 2008/0102069 describes the use of trastuzumab and pertuzumab in the treatment of HER2-positive metastatic cancer, such as breast cancer. Baselga et al., J Clin Oncol, 2007 ASCO Annual Meeting Proceedings Part I, Col. 25, No. 18S (June 20 Supplement), 2007:1004 report the treatment of patients with pre-treated HER2-positive breast cancer, which has progressed during treatment with trastuzumab, with a combination of trastuzumab and pertuzumab. Portera et al., J Clin Oncol, 2007 ASCO Annual Meeting Proceedings Part I. Vol. 25, No. 18S (June 20 Supplement), 2007:1028 evaluated the efficacy and safety of trastuzumab+pertuzumab combination therapy in HER2-positive breast cancer patients, who had progressive disease on trastuzumab-based therapy. The authors concluded that further evaluation of the efficacy of combination treatment was required to define the overall risk and benefit of this treatment regimen.

Pertuzumab has been evaluated in Phase II studies in combination with trastuzumab in patients with HER2-positive metastatic breast cancer who have previously received trastuzumab for metastatic disease. One study, conducted by the National cancer Institute (NCI), enrolled 11 patients with previously treated HER2-positive metastatic breast cancer. Two out of the 11 patients exhibited a partial response (PR) (Baselga et al., J Clin Oncol 2007 ASCO Annual Meeting Proceedings; 25:18S (June 20 Supplement): 1004).

The results of a Phase II neoadjuvant study evaluating the effect of a novel combination regimen of pertuzumab and trastuzumab plus chemotherapy (docetaxel) in women with early-stage HER2-positive breast cancer, presented at the CTRC-AACR San Antonio Breast Cancer Symposium (SABCS), Dec. 8-12, 2010, showed that the two HER2 antibodies plus docetaxel given in the neoadjuvant setting prior to surgery significantly improved the rate of complete tumor disappearance (pathological complete response rate, pCR, of 45.8 percent) in the breast by more than half compared to trastuzumab plus docetaxel (pCR of 29.0 percent), p=0.014.

The Clinical Evaluation of pertuzumab and trastuzumab (CLEOPATRA) Phase II clinical study assessed the efficacy and safety of pertuzumab plus trastuzumab plus docetaxel, as compared with placebo plus trastuzumab plus docetaxel, as first-line treatment for patients with locally recurrent, unresectable, or metastatic HER2-positive breast cancer. The combination of pertuzumab plus trastuzumab plus docetaxel, as compared with placebo plus trastuzumab plus docetaxel, when used as first-line treatment for HER2-positive metastatic breast cancer, significantly prolonged progression-free survival, with no increase in cardiac toxic effects. (Baselga et al., N Eng J Med 2012 366:2, 109-119).

The Phase II clinical study NeoSphere assessed the efficacy and safety of neoadjuvant administration of pertuzumab and trastuzumab in treatment-naïve women (patients who has not received any previous cancer therapy) with operable, locally advanced, and inflammatory breast cancer. Patients give pertuzumab and trastuzumab plus docetaxel showed a significantly improved pathological complete response rate compared with those given trastuzumab plus docetaxel, without substantial differences in tolerability (Gianni et al., Lancet Oncol 2012 13(1):25-32). Results of 5-year follow-up are reported by Gianni et al., Lancet Oncol 2016 17(6):791-800).

Patent Publications related to HER2 antibodies include: U.S. Pat. Nos. 5,677,171; 5,720,937; 5,720,954; 5,725,856; 5,770,195; 5,772,997; 6,165,464; 6,387,371; 6,399,063; 6,015,567; 6,333,169; 4,968,603; 5,821,337; 6,054,297; 6,407,213; 6,639,055; 6,719,971; 6,800,738; 5,648,237; 7,018,809; 6,267,958; 6,695,940; 6,821,515; 7,060,268; 7,682,609; 7,371,376; 6,127,526; 6,333,398; 6,797,814; 6,339,142; 6,417,335; 6,489,447; 7,074,404; 7,531,645; 7,846,441; 7,892,549; 6,573,043; 6,905,830; 7,129,840; 7,344,840; 7,468,252; 7,674,589; 6,949,245; 7,485,302; 7,498,030; 7,501,122; 7,537,931; 7,618,631; 7,862,817; 7,041,292; 6,627,196; 7,371,379; 6,632,979; 7,097,840; 7,575,748; 6,984,494; 7,279,287; 7,811,773; 7,993,834; 7,435,797; 7,850,966; 7,485,704; 7,807,799; 7,560,111; 7,879,325; 7,449,184; 7,700,299; and US 2010/0016556; US 2005/0244929; US 2001/0014326; US 2003/0202972; US 2006/0099201; US 2010/0158899; US 2011/0236383; US 2011/0033460; US 2005/0063972; US 2006/018739; US 2009/0220492; US 2003/0147884; US 2004/0037823; US 2005/0002928; US 2007/0292419; US 2008/0187533; US 2003/0152987; US 2005/0100944; US 2006/0183150; US2008/0050748; US 2010/0120053; US 2005/0244417; US 2007/0026001; US 2008/0160026; US 2008/0241146; US 2005/0208043; US 2005/0238640; US 2006/0034842; US 2006/0073143; US 2006/0193854; US 2006/0198843; US 2011/0129464; US 2007/0184055; US 2007/0269429; US 2008/0050373; US 2006/0083739; US 2009/0087432; US 2006/0210561; US 2002/0035736; US 2002/0001587; US 2008/0226659; US 2002/0090662; US 2006/0046270; US 2008/0108096; US 007/0166753; US 2008/0112958; US 2009/0239236; US 2004/008204; US 2009/0187007; US 2004/0106161; US 2011/0117096; US 2004/048525; US 2004/0258685; US 2009/0148401; US 2011/0117097; US 2006/0034840; US 2011/0064737; US 2005/0276812; US 2008/0171040; US 2009/0202536; US 2006/0013819; US 2006/0018899; US 2009/0285837; US 2011/0117097; US 2006/0088523; US 2010/0015157; US 2006/0121044; US 2008/0317753; US2006/0165702; US 2009/0081223; US 2006/0188509; US 2009/0155259; US 2011/0165157; US 2006/0204505; US 2006/0212956; US 2006/0275305; US 2007/0009976; US 2007/0020261; US 2007/0037228; US 2010/0112603; US 2006/0067930; US 2007/0224203; US 2008/0038271; US 2008/0050385; 2010/0285010; US 2008/0102069; US 2010/0008975; US 2011/0027190; US 2010/0298156; US 2009/0098135; US 2009/0148435; US 2009/0202546; US 2009/0226455; US 2009/0317387; and US 2011/0044977.

### SUMMARY OF THE INVENTION

Despite the significant advances in the past decade, patients with advanced or treatment refractory, HER2-positive, HER2-amplified, or HER2-mutated colorectal, biliary, urothelial, bladder, salivary, lung, pancreatic, ovary, prostate, or skin (apocrine) cancer have very few treatment options.

In one aspect, the invention concerns a method for the treatment of advanced colorectal cancer, comprising administering to a human patient with HER2-positive, HER2-amplified, or HER2-mutated advanced colorectal cancer an effective amount of a combination of pertuzumab and trastuzumab.

In a second aspect, the invention concerns a method for the treatment of advanced biliary cancer, comprising administering to a human patient with HER2-positive, HER2-amplified, or HER2-mutated advanced biliary cancer an effective amount of a combination of pertuzumab and trastuzumab.

In a third aspect, the invention concerns a method for the treatment of advanced urothelial cancer, comprising administering to a human patient with HER2-positive, HER2-amplified, or HER2-mutated advanced urothelial cancer an effective amount of a combination of pertuzumab and trastuzumab.

In a fourth aspect, the invention concerns a method for the treatment of advanced bladder cancer, comprising administering to a human patient with HER2-positive, HER2-amplified, or HER2-mutated advanced bladder cancer an effective amount of a combination of pertuzumab and trastuzumab. In one embodiment, the bladder cancer is urothelial bladder cancer.

In a fifth aspect, the invention concerns a method for the treatment of advanced salivary cancer, comprising administering to a human patient with HER2-positive, HER2-amplified, or HER2-mutated advanced salivary cancer an effective amount of a combination of pertuzumab and trastuzumab.

In a sixth aspect, the invention concerns a method for the treatment of advanced lung cancer, comprising administering to a human patient with HER2-positive, HER2-amplified, or HER2-mutated lung cancer an effective amount of a combination of pertuzumab and trastuzumab.

In a seventh aspect, the invention concerns a method for the treatment of advanced pancreatic cancer, comprising administering to a human patient with HER2-positive, HER2-amplified, or HER2-mutated advanced pancreatic cancer an effective amount of a combination of pertuzumab and trastuzumab.

In an eights aspect, the invention concerns a method for the treatment of advanced ovarian cancer, comprising administering to a human patient with HER2-positive, HER2-amplified, or HER2-mutated advanced ovarian cancer an effective amount of a combination of pertuzumab and trastuzumab.

In a ninths aspect, the invention concerns a method for the treatment of advanced prostate cancer, comprising administering to a human patient with HER2-positive, HER2-amplified, or HER2-mutated advanced prostate cancer an effective amount of a combination of pertuzumab and trastuzumab.

In a tenth aspect, the invention concerns a method for the treatment of advanced skin cancer, comprising administering to a human patient with HER2-positive, HER2-amplified, or HER2-mutated advanced skin cancer an effective amount of a combination of pertuzumab and trastuzumab.

In all aspects, if the cancer is HER2-positive, the HER2 expression level may, for example, be IHC 2+ or 3+.

In all aspects, if the cancer is HER2-amplified, HER2 amplification may, for example, be determined by fluorescence in situ hybridization (FISH).

In all aspects, if the cancer is HER2-mutated, mutations can, for example be detected by next generation sequencing (NGS) or real-time polymerase chain reaction (RT-PCR).

In certain embodiments, the HER2 mutation is selected from the group consisting of insertions within exon 20 of HER2, deletions around amino acid residues 755-759 of HER2, G309A, G309E, S310F, D769H, D769Y, V777L, P780-Y781insGSP, V8421I, R896C and other putative activating mutations found two or more unique specimens.

The advanced cancer may be locally advanced or metastatic.

In other embodiments, the cancer is refractory to another treatment regimen. Thus, the cancer may be chemotherapy-resistant, including platinum resistance.

In other embodiments, the patient treated with pertuzumab plus trastuzumab received one to five rounds of prior treatments for treating the cancer.

In various embodiments, the prior treatments may comprise chemotherapy, and/or HER2-directed therapy.

In other embodiments, at least one of such prior treatments administered in the advanced stage.

The prior treatment(s) may include neoadjuvant treatment and/or adjuvant treatment.

In certain embodiments, the patient's cancer is resistant to at least one of prior treatments.

In a further embodiment, the combination of pertuzumab and trastuzumab is administered in the absence of other anti-cancer drug(s).

In a still further embodiment, the combination of pertuzumab and trastuzumab is administered in the absence of chemotherapy.

In a different embodiment, the combination of pertuzumab and trastuzumab is administered in the absence of another HER2 directed therapy.

In yet another embodiment, the treatment consists essentially of combined administration of a combination of pertuzumab and trastuzumab.

The treatment methods of the present invention may result in improved overall response rate (ORR) relative to administration of pertuzumab or trastuzumab as a single agent and/or in improved partial response (PR) relative to administration of pertuzumab or trastuzumab as a single agent and/or in improved complete response (CR) relative to administration of pertuzumab or trastuzumab as a single agent.

In other embodiments, the combined administration of pertuzumab and trastuzumab extends survival of said patient relative to administration of pertuzumab or trastuzumab as a single agent.

In further embodiments, the combined administration of pertuzumab and trastuzumab extends progression-free survival (PFS) of the patient.

In still further embodiments, the combined administration of pertuzumab and trastuzumab extends overall survival (OS) of the patient.

In another embodiment, the combined administration of pertuzumab and trastuzumab results in a synergistic effect.

In yet another embodiment, the combined administration of pertuzumab and trastuzumab does not result in an increase of side-effects relative to monotherapy with pertuzumab or trastuzumab.

In a further embodiment, the combined administration of pertuzumab and trastuzumab does not result in an increase of cardiac-side-effects relative to monotherapy with pertuzumab or trastuzumab.

In another aspect, the invention concerns an article of manufacture comprising a vial with pertuzumab and a package insert, wherein the package insert provides instructions to administer said pertuzumab as hereinabove described.

In a further aspect the invention concerns a composition of pertuzumab for use, in combination with trastuzumab, for the treatment of a human patient with HER2-positive, HER2-amplified, or HER2-mutated advanced colorectal cancer.

In a second aspect, the invention concerns a composition of pertuzumab for use, in combination with trastuzumab, for the treatment of a human patient with HER2-positive, HER2-amplified, or HER2-mutated advanced biliary cancer.

In a third aspect, the invention concerns a composition of pertuzumab for use, in combination with trastuzumab, for the treatment of a human patient with HER2-positive, HER2-amplified, or HER2-mutated advanced urothelial cancer.

In a fourth aspect, the invention concerns a composition of pertuzumab for use, in combination with trastuzumab, for the treatment of a human patient with HER2-positive, HER2-amplified, or HER2-mutated advanced bladder cancer. In one embodiment, the bladder cancer is urothelial bladder cancer.

In a fifth aspect, the invention concerns a composition of pertuzumab for use, in combination with trastuzumab, for the treatment of a human patient with HER2-positive, HER2-amplified, or HER2-mutated advanced salivary cancer.

In a sixth aspect, the invention concerns a composition of pertuzumab for use, in combination with trastuzumab, for the treatment of a human patient with HER2-positive, HER2-amplified, or HER2-mutated lung cancer.

In a seventh aspect, the invention concerns a composition of pertuzumab for use, in combination with trastuzumab, for the treatment of HER2-positive, HER2-amplified, or HER2-mutated pancreatic cancer.

In an eights aspect, the invention concerns a composition of pertuzumab for use, in combination with trastuzumab, for the treatment of HER2-positive, HER2-amplified, or HER2-mutated ovarian cancer.

In a ninths aspect, the invention concerns a composition of pertuzumab for use, in combination with trastuzumab, for the treatment of HER2-positive, HER2-amplified, or HER2-mutated prostate cancer.

In a tenth aspect, the invention concerns a composition of pertuzumab for use, in combination with trastuzumab, for the treatment of HER2-positive, HER2-amplified, or HER2-mutated skin (apocrine) cancer.

In another aspect, the invention concerns the use of pertuzumab in the preparation of a medicament for the treatment of a human patient with HER2-positive, HER2-amplified, or HER2-mutated advanced colorectal cancer, in combination with trastuzumab.

In second aspect, the invention concerns the use of pertuzumab in the preparation of a medicament for the treatment of a human patient with HER2-positive, HER2-amplified, or HER2-mutated advanced biliary cancer, in combination with trastuzumab.

In a third aspect, the invention concerns the use of pertuzumab in the preparation of a medicament for the treatment of a human patient with HER2-positive, HER2-amplified, or HER2-mutated advanced urothelial cancer, in combination with trastuzumab.

In a fourth aspect, the invention concerns the use of pertuzumab in the preparation of a medicament for the treatment of a human patient with HER2-positive, HER2-amplified, or HER2-mutated advanced bladder cancer, in combination with trastuzumab. In one embodiment, the bladder cancer is urothelial bladder cancer.

In a fifth aspect, the invention concerns the use of pertuzumab in the preparation of a medicament for the treatment of a human patient with HER2-positive, HER2-amplified, or HER2-mutated advanced salivary cancer, in combination with trastuzumab.

In a sixth aspect, the invention concerns the use of pertuzumab in the preparation of a medicament for the treatment of a human patient with HER2-positive, HER2-amplified, or HER2-mutated advanced lung cancer, in combination with trastuzumab.

In a seventh aspect, the invention concerns the use of pertuzumab in the preparation of a medicament for the treatment of a human patient with HER2-positive, HER2-amplified, or HER2-mutated advanced pancreatic cancer, in combination with trastuzumab.

In an eights aspect, the invention concerns the use of pertuzumab in the preparation of a medicament for the treatment of a human patient with HER2-positive, HER2-amplified, or HER2-mutated advanced ovarian cancer, in combination with trastuzumab.

In a ninths aspect, the invention concerns the use of pertuzumab in the preparation of a medicament for the treatment of a human patient with HER2-positive, HER2-amplified, or HER2-mutated advanced prostate cancer, in combination with trastuzumab.

In a tenth aspect, the invention concerns the use of pertuzumab in the preparation of a medicament for the treatment of a human patient with HER2-positive, HER2-amplified, or HER2-mutated advanced skin (apocrine) cancer, in combination with trastuzumab.

These and further aspects will be apparent from the disclosure herein, including the Examples.

### BRIEF DESCRIPTION OF THE DRAWINGS

FIG. 1 provides a schematic of the HER2 protein structure, and amino acid sequences for Domains I-IV (SEQ ID Nos.1-4, respectively) of the extracellular domain thereof.

FIGS. 2A and 2B depict alignments of the amino acid sequences of the variable light (V_{L}) (FIG. 2A) and variable heavy (V_{H}) (FIG. 2B) domains of murine monoclonal antibody 2C4 (SEQ ID Nos. 5 and 6, respectively); V_{L} and V_{H} domains of variant 574/pertuzumab (SEQ ID NOs. 7 and 8, respectively), and human V_{L} and V_{H} consensus frameworks (hum id, light kappa subgroup I; humIII, heavy subgroup III) (SEQ ID Nos. 9 and 10, respectively). Asterisks identify differences between variable domains of pertuzumab and murine monoclonal antibody 2C4 or between variable domains of pertuzumab and the human framework. Complementarity Determining Regions (CDRs) are in brackets.

FIGS. 3A and 3B show the amino acid sequences of pertuzumab light chain (FIG. 3A; SEQ ID NO. 11) and heavy chain (FIG. 3B; SEQ ID No. 12). CDRs are shown in bold. Calculated molecular mass of the light chain and heavy chain are 23,526.22 Da and 49,216.56 Da (cysteines in reduced form). The carbohydrate moiety is attached to Asn 299 of the heavy chain.

FIGS. 4A and 4B show the amino acid sequences of trastuzumab light chain (FIG. 4A; SEQ ID NO. 13) and heavy chain (FIG. 4B; SEQ ID NO. 14), respectively. Boundaries of the variable light and variable heavy domains are indicated by arrows.

FIGS. 5A and 5B depict a variant pertuzumab light chain sequence (FIG. 5A; SEQ ID NO. 15) and a variant pertuzumab heavy chain sequence (FIG. 5B; SEQ ID NO. 16), respectively.

FIG. 6 shows the main study schema of the MyPathway clinical trial.

FIG. 7 shows the study design for the study described in Example 1.

FIG. 8 shows the algorithm for addressing asymptomatic decline in LVEF.

FIG. 9 shows time of treatment for patients with HER2-amplified/overexpressed mCRC (n=34). + indicates that treatment is ongoing; K indicates the patient has a KRAS mutation; dashed line indicates 4 months.

FIG. 10 shows the best percent change from baseline in target lesion size in patients with HER2-amplified/overexpressed mRCR (n=31). + indicates that the treatment is ongoing; K indicates the patient has a KRAS mutation. ^{a} Three patients are excluded from this plot: 2 patients (including 1 with KRAS mutation) show discontinued treatment due to clinical progression without a post-baseline tumor assessment, and 1 who discontinued treatment due to a new lesion and who was missing three quarters of the target lesion assessments. ^{b} “Percent change from baseline” represents the maximum reduction/minimum increase in the target lesion size, or the appearance of one or more new lesions. Patients with 30% decrease in the target lesion size qualify for PR; patients with at least a 20% increase in target lesion size, or the appearance of one or more new lesions qualify for PD.

FIG. 11 shows PFS in patients with HER2-amplified/overexpressed mCRC.

FIG. 12 shows OS in patients with HER2-amplified/overexpressed mCRC.

FIG. 13 shows a waterfall plot of treatment response in patients with HER2-amplified/overexpressing biliary cancer (N=8)

FIG. 14 shows a waterfall plot of treatment response in patients with HER2-amplified/overexpressing bladder cancer patients (N=8).

FIG. 15 shows time on treatment in patients with HER2-amplified/overexpressed or HER2-mutated metastatic urothelial cancer (mUC) (n=12).

FIG. 16 shows the best percent change from baseline in target lesion size by patients.

FIGS. 17A to C show CT scans of a complete tumor response in a patient with HER2-positive mUC at different time points.

A) April 2015: Baseline scan. The largest collection of metastases was found anterior to the mid transverse colon, measuring 3.5 cm×1.6 cm.

B) June 2015: First post-baseline scan shows a reduction in the omental implants since the last CT and no measurable disease. Arrow demarcates reduction of soft tissue mass within the omentum with only a linear strand of soft tissue remaining.

C) December 2016: No evidence for recurrent or metastatic disease.

### DETAILED DESCRIPTION OF THE PREFERRED EMBODIMENTS

### I. Definitions

“Survival” refers to the patient remaining alive, and includes overall survival (OS) as well as progression free survival (PFS).

“Overall survival” or “OS” refers to the patient remaining alive for a defined period of time, such as 1 year, 5 years, 12 years, 10 years, 15 years, etc. from the time of diagnosis or treatment. For the purposes of the clinical trial described in the example, overall survival (OS) is defined as the time from the date of randomization of patient population to the date of death from any cause.

“Progression free survival” or “PFS” refers to the patient remaining alive, without the cancer progressing or getting worse. For the purpose of the clinical trial described in the example, progression free survival (PFS) is defined as the time from randomization of study population to the first documented progressive disease, or unmanageable toxicity, or death from any cause, whichever occurs first. Disease progression can be documented by any clinically accepted methods, such as, for example, radiographical progressive disease, as determined by Response Evaluation Criteria in Solid Tumors (RECIST) (Therasse et al., J Natl Ca Inst 2000; 92(3):205-216), carcinomatous meningitis diagnosed by cytologic evaluation of cerebral spinal fluid, and/or medical photography to monitor chest wall recurrences of subcutaneous lesions.

By “extending survival” is meant increasing overall or progression free survival in a patient treated in accordance with the present invention relative to an untreated patient and/or relative to a patient treated with one or more approved anti-tumor agents, but not receiving treatment in accordance with the present invention. In a particular example, “extending survival” means extending progression-free survival (PFS) and/or overall survival (OS) of cancer patients receiving the combination therapy of the present invention (e.g. treatment with a combination of pertuzumab, trastuzumab and a chemotherapy) relative to patients treated with trastuzumab and the chemotherapy only. In another particular example, “extending survival” means extending progression-free survival (PFS) and/or overall survival (OS) of cancer patients receiving the combination therapy of the present invention (e.g. treatment with a combination of pertuzumab, trastuzumab and a chemotherapy) relative to patients treated with pertuzumab and the chemotherapy only.

An “objective response” or “OR” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

A “HER receptor” is a receptor protein tyrosine kinase which belongs to the HER receptor family and includes EGFR, HER2, HER3 and HER4 receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. Preferably the HER receptor is native sequence human HER receptor.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat p185^{neu}. Preferred HER2 is native sequence human HER2.

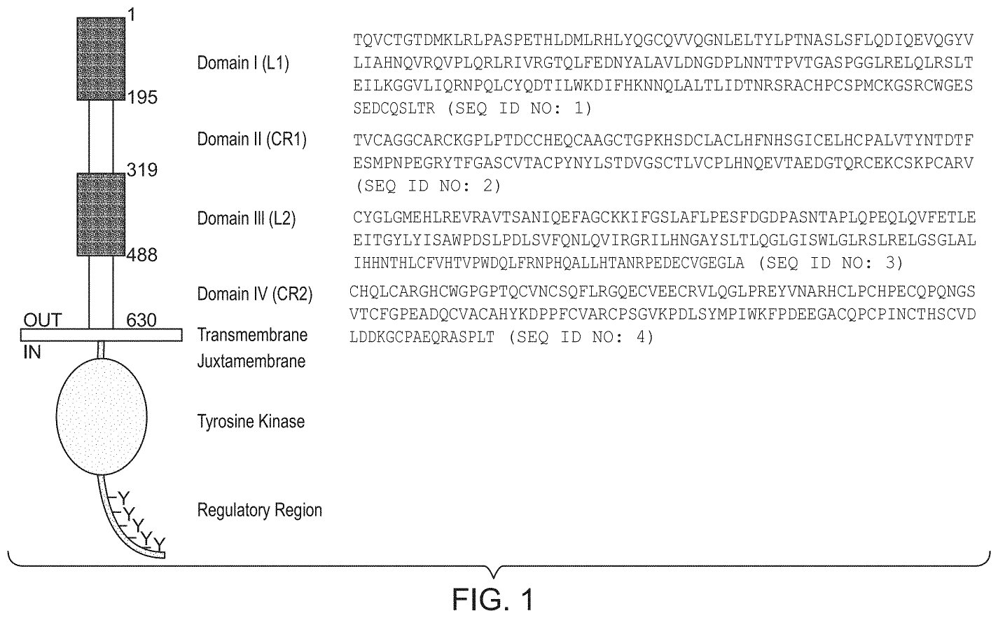

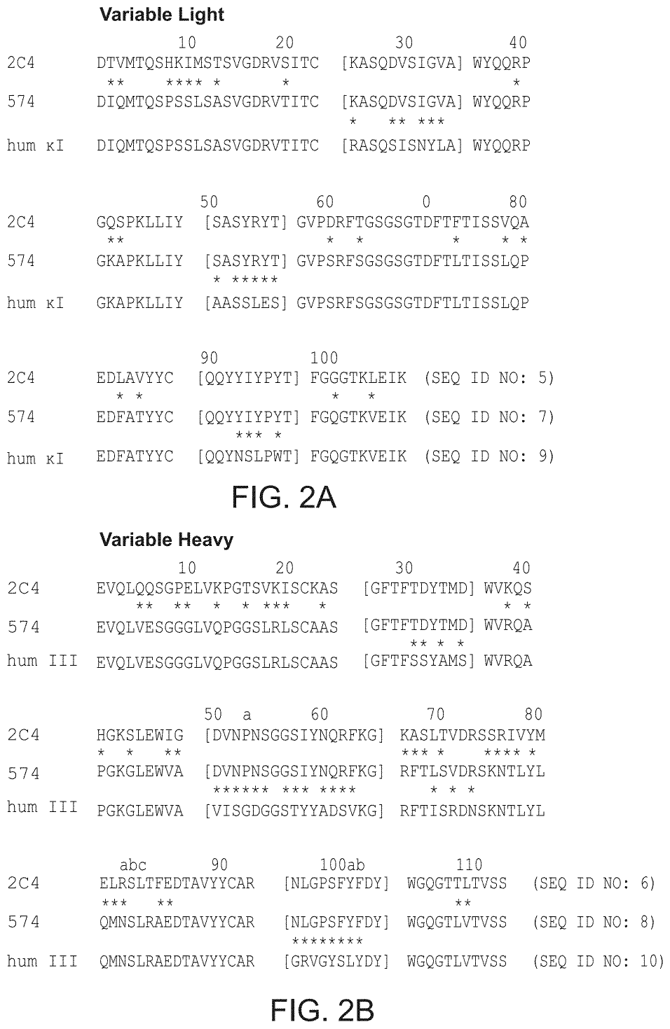

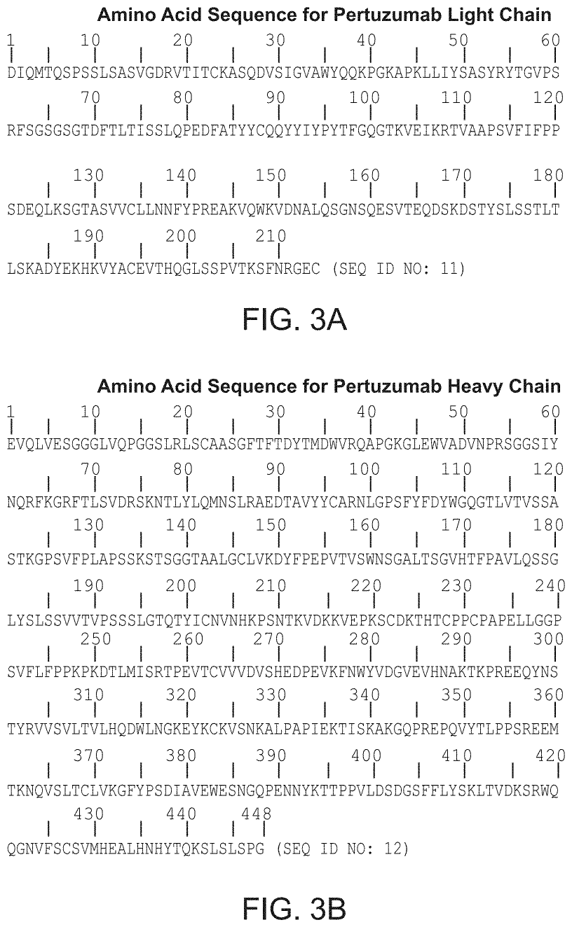

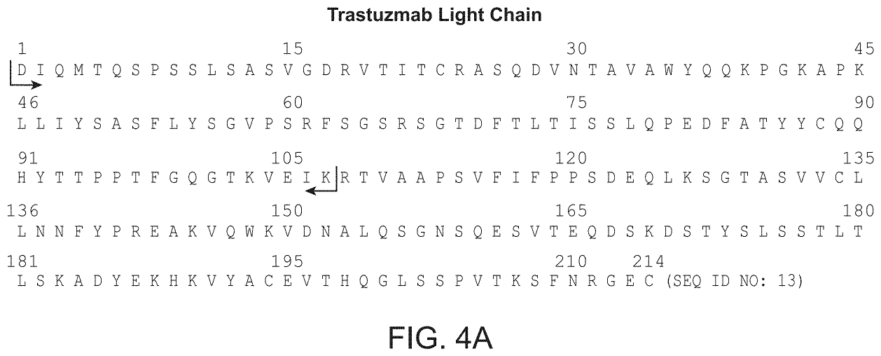

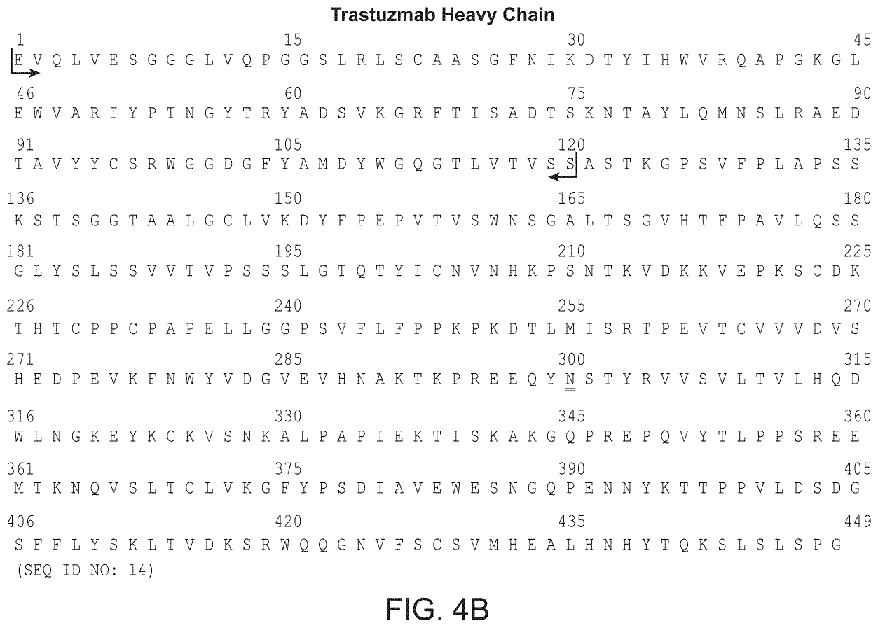

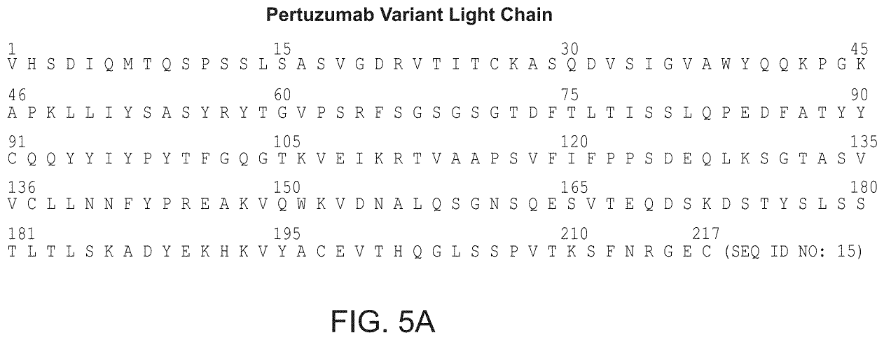

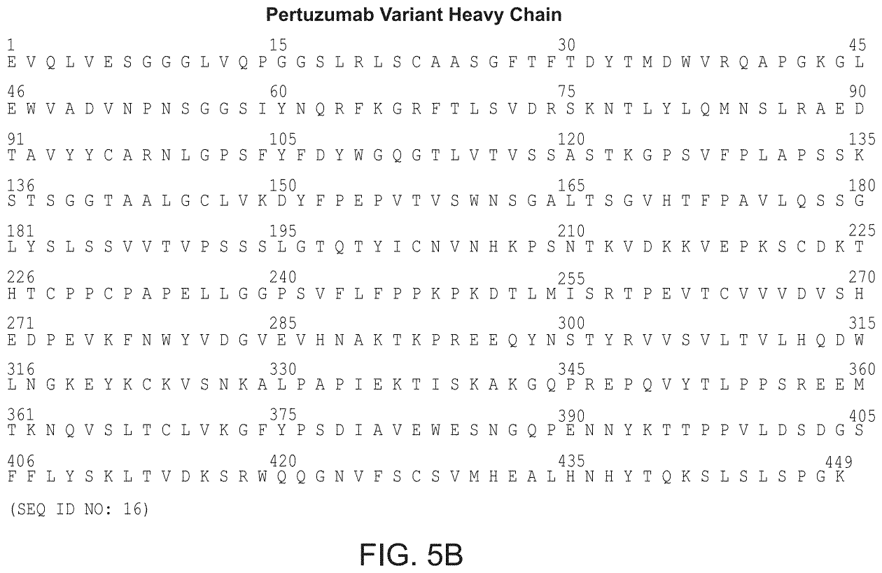

Herein, “HER2 extracellular domain” or “HER2 ECD” refers to a domain of HER2 that is outside of a cell, either anchored to a cell membrane, or in circulation, including fragments thereof. The amino acid sequence of HER2 is shown in FIG. 1. In one embodiment, the extracellular domain of HER2 may comprise four domains: “Domain I” (amino acid residues from about 1-195; SEQ ID NO:1), “Domain II” (amino acid residues from about 196-319; SEQ ID NO:2), “Domain III” (amino acid residues from about 320-488: SEQ ID NO:3), and “Domain IV” (amino acid residues from about 489-630; SEQ ID NO:4) (residue numbering without signal peptide). See Garrett et al. Mol. Cell. 11: 495-505 (2003), Cho et al. Nature 421: 756-760 (2003), Franklin et al. Cancer Cell 5:317-328 (2004), and Plowman et al. Proc. Natl. Acad. Sci. 90:1746-1750 (1993), as well as FIG. 1 herein.

“HER3” or “ErbB3” herein refer to the receptor as disclosed, for example, in U.S. Pat. Nos. 5,183,884 and 5,480,968 as well as Kraus et al. PNAS (USA) 86:9193-9197 (1989).

A “low HER3” cancer is one which expresses HER3 at a level less than the median level for HER3 expression in the cancer type. In one embodiment, the low HER3 cancer is epithelial ovarian, peritoneal, or fallopian tube cancer. HER3 DNA, protein, and/or mRNA level in the cancer can be evaluated to determine whether the cancer is a low HER3 cancer. See, for example, U.S. Pat. No. 7,981,418 for additional information about low HER3 cancer. Optionally, a HER3 mRNA expression assay is performed in order to determine that the cancer is a low HER3 cancer. In one embodiment, HER3 mRNA level in the cancer is evaluated, e.g. using polymerase chain reaction (PCR), such as quantitative reverse transcription PCR (qRT-PCR). Optionally, the cancer expresses HER3 at a concentration ratio equal or lower than about 2.81 as assessed qRT-PCR, e.g. using a COBAS z480® instrument.

A “HER dimer” herein is a noncovalently associated dimer comprising at least two HER receptors. Such complexes may form when a cell expressing two or more HER receptors is exposed to an HER ligand and can be isolated by immunoprecipitation and analyzed by SDS-PAGE as described in Sliwkowski et al., J. Biol. Chem., 269(20):14661-14665 (1994), for example. Other proteins, such as a cytokine receptor subunit (e.g. gp130) may be associated with the dimer. Preferably, the HER dimer comprises HER2.

A “HER heterodimer” herein is a noncovalently associated heterodimer comprising at least two different HER receptors, such as EGFR-HER2, HER2-HER3 or HER2-HER4 heterodimers.

A “HER antibody” is an antibody that binds to a HER receptor. Optionally, the HER antibody further interferes with HER activation or function. Preferably, the HER antibody binds to the HER2 receptor. HER2 antibodies of interest herein are pertuzumab and trastuzumab.

“HER activation” refers to activation, or phosphorylation, of any one or more HER receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

“Phosphorylation” refers to the addition of one or more phosphate group(s) to a protein, such as a HER receptor, or substrate thereof.

An antibody which “inhibits HER dimerization” is an antibody which inhibits, or interferes with, formation of a HER dimer. Preferably, such an antibody binds to HER2 at the heterodimeric binding site thereof. The most preferred dimerization inhibiting antibody herein is pertuzumab or MAb 2C4. Other examples of antibodies which inhibit HER dimerization include antibodies which bind to EGFR and inhibit dimerization thereof with one or more other HER receptors (for example EGFR monoclonal antibody 806, MAb 806, which binds to activated or “untethered” EGFR; see Johns et al., J. Biol. Chem. 279(29):30375-30384 (2004)); antibodies which bind to HER3 and inhibit dimerization thereof with one or more other HER receptors; and antibodies which bind to HER4 and inhibit dimerization thereof with one or more other HER receptors.

A “HER2 dimerization inhibitor” is an agent that inhibits formation of a dimer or heterodimer comprising HER2.

A “heterodimeric binding site” on HER2, refers to a region in the extracellular domain of HER2 that contacts, or interfaces with, a region in the extracellular domain of EGFR, HER3 or HER4 upon formation of a dimer therewith. The region is found in Domain II of HER2 (SEQ ID NO: 15). Franklin et al. Cancer Cell 5:317-328 (2004).

A HER2 antibody that “binds to a heterodimeric binding site” of HER2, binds to residues in Domain II (SEQ ID NO: 2) and optionally also binds to residues in other of the domains of the HER2 extracellular domain, such as domains I and III, SEQ ID NOs: 1 and 3), and can sterically hinder, at least to some extent, formation of a HER2-EGFR, HER2-HER3, or HER2-HER4 heterodimer. Franklin et al. Cancer Cell 5:317-328 (2004) characterize the HER2-pertuzumab crystal structure, deposited with the RCSB Protein Data Bank (ID Code IS78), illustrating an exemplary antibody that binds to the heterodimeric binding site of HER2.

An antibody that “binds to domain II” of HER2 binds to residues in domain II (SEQ ID NO: 2) and optionally residues in other domain(s) of HER2, such as domains I and III (SEQ ID NOs: 1 and 3, respectively). Preferably the antibody that binds to domain II binds to the junction between domains I, II and III of HER2.

For the purposes herein, “pertuzumab” and “rhuMAb 2C4”, which are used interchangeably, refer to an antibody comprising the variable light and variable heavy amino acid sequences in SEQ ID NOs: 7 and 8, respectively. Where pertuzumab is an intact antibody, it preferably comprises an IgG1 antibody; in one embodiment comprising the light chain amino acid sequence in SEQ ID NO: 11 or 15, and heavy chain amino acid sequence in SEQ ID NO: 12 or 16. The antibody is optionally produced by recombinant Chinese Hamster Ovary (CHO) cells. The terms “pertuzumab” and “rhuMAb 2C4” herein cover biosimilar versions of the drug with the United States Adopted Name (USAN) or International Nonproprietary Name (INN): pertuzumab.

For the purposes herein, “trastuzumab” and rhuMAb4D5”, which are used interchangeably, refer to an antibody comprising the variable light and variable heavy amino acid sequences from within SEQ ID Nos: 13 and 14, respectively. Where trastuzumab is an intact antibody, it preferably comprises an IgG1 antibody; in one embodiment comprising the light chain amino acid sequence of SEQ ID NO: 13 and the heavy chain amino acid sequence of SEQ ID NO: 14. The antibody is optionally produced by Chinese Hamster Ovary (CHO) cells. The terms “trastuzumab” and “rhuMAb4D5” herein cover biosimilar versions of the drug with the United States Adopted Name (USAN) or International Nonproprietary Name (INN): trastuzumab.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). Humanized HER2 antibodies specifically include trastuzumab (HERCEPTIN®) as described in Table 3 of U.S. Pat. No. 5,821,337 expressly incorporated herein by reference and as defined herein; and humanized 2C4 antibodies such as pertuzumab as described and defined herein.

An “intact antibody” herein is one which comprises two antigen binding regions, and an Fc region. Preferably, the intact antibody has a functional Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into Asubclasses@ (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

A “naked antibody” is an antibody that is not conjugated to a heterologous molecule, such as a cytotoxic moiety or radiolabel.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “deamidated” antibody is one in which one or more asparagine residues thereof has been derivitized, e.g. to an aspartic acid, a succinimide, or an iso-aspartic acid.

The terms “cancer” and “cancerous” refer to or describe the physiological condition in mammals that is typically characterized by unregulated cell growth. The term “cancer” specifically includes, without limitation, HER2-positive, HER2-amplified and/or HER2-mutated cancer, such as colorectal cancer, biliary cancer, urothelial cancer, bladder cancer, salivary cancer, lung cancer, e.g. non-small-cell lung (NSCLC) cancer, pancreatic, ovary, prostate, and skin (apocrine) cancer.

“Colorectal cancer” is a cancer that develops in any part of the colon and/or the rectum. The term specifically includes advanced, including metastatic and locally advanced colorectal cancer, inoperable (non-resectable) colorectal cancer, colorectal cancer not amenable to curative therapy, and post-operatively recurrent advanced colorectal cancer, as well as treatment-resistance colorectal cancer, including, without limitation, histologically confirmed adenocarcinomas, primary colorectal lymphomas, gastrointestinal stromal tumors, leiomyosarcomas, carcinoid tumors and melanomas. Adenocarcinomas are the predominant form of colorectal cancer.

“Bladder cancer” specifically includes all types and stages of bladder cancer, specifically including, without limitation, metastatic and locally advanced bladder cancer, inoperable (non-resectable) bladder cancer, bladder cancer not amenable to curative therapy, and post-operatively recurrent advanced bladder cancer, as well as treatment-resistance bladder cancer. The term specifically includes, without limitation, urothelial carcinoma, squamous cell carcinoma, and adenocarcinoma, and non-invasive, non-muscle invasive, and muscle invasive forms of bladder cancer. In one embodiment, the bladder cancer is urothelial bladder cancer.

“Biliary cancer” includes all cancers of the biliary duct, specifically including, without limitation, metastatic and locally advanced biliary cancer, inoperable (non-resectable) biliary cancer, biliary cancer not amenable to curative therapy, and post-operatively recurrent advanced biliary cancer, as well as treatment-resistance biliary cancer, including, without limitation, intrahepatic bile duct cancers.

“Gastric cancer” specifically includes metastatic or locally advanced non-resectable gastric cancer, including, without limitation, histologically confirmed adenocarcinoma of the stomach or gastroesophageal junction with inoperable (non-resectable) locally advanced or metastatic disease, not amenable to curative therapy, and post-operatively recurrent advanced gastric cancer, such as adenocarcinoma of the stomach or gastroesophageal junction, when the intent of the surgery was to cure the disease.

An “advanced” cancer is one which has spread outside the site or organ of origin, either by local invasion (“locally advanced”) or metastasis (“metastatic”). Accordingly, the term “advanced” cancer includes both locally advanced and metastatic disease.

“Metastatic” cancer refers to cancer which has spread from one part of the body (e.g. the breast) to another part of the body.

A “refractory” cancer is one which progresses even though an anti-tumor agent, such as a chemotherapy or biologic therapy, such as immunotherapy, is being administered to the cancer patient. An example of a refractory cancer is one which is platinum refractory.

A “recurrent” cancer is one which has regrown, either at the initial site or at a distant site, after a response to initial therapy, such as surgery.

A “locally recurrent” cancer is cancer that returns after treatment in the same place as a previously treated cancer.

A “non-resectable” or “unresectable” cancer is not able to be removed (resected) by surgery.

“Early-stage breast cancer” herein refers to breast cancer that has not spread beyond the breast or the axillary lymph nodes. Such cancer is generally treated with neoadjuvant or adjuvant therapy.

“Neoadjuvant therapy” or “neoadjuvant treatment” or “neoadjuvant administration” refers to systemic therapy given prior to surgery.

“Adjuvant therapy” or “adjuvant treatment” or “adjuvant administration” refers to systemic therapy given after surgery.

Herein, a “patient” or “subject” is a human patient. The patient may be a “cancer patient,” i.e. one who is suffering or at risk for suffering from one or more symptoms of cancer, in particular breast cancer.

A “patient population” refers to a group of cancer patients. Such populations can be used to demonstrate statistically significant efficacy and/or safety of a drug, such as pertuzumab and/or trastuzumab.

A “relapsed” patient is one who has signs or symptoms of cancer after remission. Optionally, the patient has relapsed after adjuvant or neoadjuvant therapy.

A cancer or biological sample which “displays HER expression, amplification, or activation” is one which, in a diagnostic test, expresses (including overexpresses) a HER receptor, has amplified HER gene, and/or otherwise demonstrates activation or phosphorylation of a HER receptor.

A cancer or biological sample which “displays HER activation” is one which, in a diagnostic test, demonstrates activation or phosphorylation of a HER receptor. Such activation can be determined directly (e.g. by measuring HER phosphorylation by ELISA) or indirectly (e.g. by gene expression profiling or by detecting HER heterodimers, as described herein).

A cancer cell with “HER receptor overexpression or amplification” is one which has significantly higher levels of a HER receptor protein or gene compared to a noncancerous cell of the same tissue type. Such overexpression may be caused by gene amplification or by increased transcription or translation. HER receptor overexpression or amplification may be determined in a diagnostic or prognostic assay by evaluating increased levels of the HER protein present on the surface of a cell (e.g. via an immunohistochemistry assay; IHC). Alternatively, or additionally, one may measure levels of HER-encoding nucleic acid in the cell, e.g. via in situ hybridization (ISH), including fluorescent in situ hybridization (FISH; see WO98/45479 published October, 1998) and chromogenic in situ hybridization (CISH; see, e.g. Tanner et al., Am. J. Pathol. 157(5): 1467-1472 (2000); Bella et al., J. Clin. Oncol. 26: (May 20 suppl; abstr 22147) (2008)), southern blotting, or polymerase chain reaction (PCR) techniques, such as quantitative real time PCR (qRT-PCR). One may also study HER receptor overexpression or amplification by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

A “HER2-positive” cancer comprises cancer cells which have higher than normal levels of HER2. Examples of HER2-positive cancer include HER2-positive colorectal cancer, HER2-positive biliary cancer, HER2-positive urothelial cancer, and HER2-positive bladder cancer. Optionally, HER2-positive cancer has an immunohistochemistry (IHC) score of 2+ or 3+ and/or an in situ hybridization (ISH) amplification ratio ≥2.0. Optionally, the HER2-positive cancer has HER2 amplification characterized by a HER2/CEP17 ratio >2.0 (fluorescent or chromogenic in situ hybridization [FISH or CISH]), or a gene copy number >6 (FISH/CISH or next-generation sequencing [NGS]).

A “HER2-mutated” cancer comprises cancer cells with a HER2-activating mutation, including kinase domain mutations, which can, for example, be identified by next generation sequencing (NGS) or real-time polymerase chain reaction (RT-PCR). “HER2-mutated” cancer specifically includes cancer characterized by insertions in exon 20 of HER2, deletions around amino acid residues 755-759 of HER2, any of the mutations G309A, G309E, S310F, D769H, D769Y, V777L, P780-Y781insGSP, V842I, R896C (Bose et al., Cancer Discov 2013; 3:1-14), as well as previously reported identical non-synonymous putative activating mutations (or indels) in COSMIC database found in two or more unique specimens. For further details see, e.g. Stephens et al., Nature 2004; 431:525-6; Shigematsu et al., Cancer Res 2005; 65:1642-6; Buttitta et al., Int J Cancer 2006; 119:2586-91; Li et al., Oncogene 2008; 27:4702-11; Sequist et al., J Clin Oncol 2010; 28:3076-83; Arcila et al., Clin Cancer Res 2012; 18:4910-8; Greulich et al., Proc Natl Acad Sci USA 2012; 109:14476-81; and Herter-Sprie et al., Front Oncol 2013; 3:1-10. The HER2-mutated cancer may, for example, be HER2-mutated colorectal cancer, HER2-mutated biliary cancer, HER2-mutated urothelial cancer, HER2-mutated bladder cancer, HER2-mutated salivary cancer, or HER2-mutated lung cancer.

Herein, an “anti-tumor agent” refers to a drug used to treat cancer. Non-limiting examples of anti-tumor agents herein include chemotherapy agents, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

The “epitope 2C4” is the region in the extracellular domain of HER2 to which the antibody 2C4 binds. In order to screen for antibodies which bind essentially to the 2C4 epitope, a routine cross-blocking assay such as that described in Antibodies, A Laboratory Manual, Cold Spring Harbor Laboratory, Ed Harlow and David Lane (1988), can be performed. Preferably the antibody blocks 2C4's binding to HER2 by about 50% or more. Alternatively, epitope mapping can be performed to assess whether the antibody binds essentially to the 2C4 epitope of HER2. Epitope 2C4 comprises residues from Domain II (SEQ ID NO: 2) in the extracellular domain of HER2. 2C4 and pertuzumab binds to the extracellular domain of HER2 at the junction of domains I, II and III (SEQ ID NOs: 1, 2, and 3, respectively). Franklin et al. Cancer Cell 5:317-328 (2004).

The “epitope 4D5” is the region in the extracellular domain of HER2 to which the antibody 4D5 (ATCC CRL 10463) and trastuzumab bind. This epitope is close to the transmembrane domain of HER2, and within Domain IV of HER2 (SEQ ID NO: 4). To screen for antibodies which bind essentially to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds essentially to the 4D5 epitope of HER2 (e.g. any one or more residues in the region from about residue 529 to about residue 625, inclusive of the HER2 ECD, residue numbering including signal peptide).

“Treatment” refers to both therapeutic treatment and prophylactic or preventative measures. Those in need of treatment include those already with cancer as well as those in which cancer is to be prevented. Hence, the patient to be treated herein may have been diagnosed as having cancer or may be predisposed or susceptible to cancer.

The term “effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapy” is use of a chemical compound useful in the treatment of cancer. Examples of chemotherapeutic agents, used in chemotherapy, include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)) and anthracyclines such as annamycin, AD 32, alcarubicin, daunorubicin, doxorubicin, dexrazoxane, DX-52-1, epirubicin, GPX-100, idarubicin, valrubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX®, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK7 polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxanes; chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “taxane” is a chemotherapy which inhibits mitosis and interferes with microtubules. Examples of taxanes include Paclitaxel (TAXOL®; Bristol-Myers Squibb Oncology, Princeton, N.J.); cremophor-free, albumin-engineered nanoparticle formulation of paclitaxel or nab-paclitaxel (ABRAXANE™; American Pharmaceutical Partners, Schaumberg, Ill.); and Docetaxel (TAXOTERE®; Rhone-Poulenc Rorer, Antony, France).

An “anthracycline” is a type of antibiotic that comes from the fungus Streptococcus peucetius, examples include: daunorubicin, doxorubicin, epirubicin, and any other anthracycline chemotherapeutic agents, including those listed before.

“Anthracycline-based chemotherapy” refers to a chemotherapy regimen that consists of or includes one or more anthracycline. Examples include, without limitation, 5-FU, epirubicin, and cyclophosphamide (FEC); 5-FU, doxorubicin, and cyclophosphamide (FAC); doxorubicin and cyclophosphamide (AC); epirubicin and cyclophosphamide (EC); dose-dense doxorubicin and cyclophosphamide (ddAC), and the like.

For the purposes herein, “carboplatin-based chemotherapy” refers to a chemotherapy regimen that consists of or includes one or more Carboplatins. An example is TCH (Docetaxel/TAXOL®, Carboplatin, and trastuzumab/HERCEPTIN®).

An “aromatase inhibitor” inhibits the enzyme aromatase, which regulates estrogen production in the adrenal glands. Examples of aromatase inhibitors include: 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In one embodiment, the aromatase inhibitor herein is letrozole or anastrozole.

An “antimetabolite chemotherapy” is use of an agent which is structurally similar to a metabolite, but cannot be used by the body in a productive manner. Many antimetabolite chemotherapy interferes with the production of the nucleic acids, RNA and DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc.

By “chemotherapy-resistant” cancer is meant that the cancer patient has progressed while receiving a chemotherapy regimen (i.e. the patient is “chemotherapy refractory”), or the patient has progressed within 12 months (for instance, within 6 months) after completing a chemotherapy regimen.

The term “platin” is used herein to refer to platinum based chemotherapy, including, without limitation, cisplatin, carboplatin, and oxaliplatin.

The term “fluoropyrimidine” is used herein to refer to an antimetabolite chemotherapy, including, without limitation, capecitabine, floxuridine, and fluorouracil (5-FU).

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinyl chloride.

A “vial” is a container suitable for holding a liquid or lyophilized preparation. In one embodiment, the vial is a single-use vial, e.g. a 20-cc single-use vial with a stopper.

A “package insert” is a leaflet that, by order of the Food and Drug Administration (FDA) or other Regulatory Authority, must be placed inside the package of every prescription drug. The leaflet generally includes the trademark for the drug, its generic name, and its mechanism of action; states its indications, contraindications, warnings, precautions, adverse effects, and dosage forms; and includes instructions for the recommended dose, time, and route of administration.

The expression “safety data” concerns the data obtained in a controlled clinical trial showing the prevalence and severity of adverse events to guide the user regarding the safety of the drug, including guidance on how to monitor and prevent adverse reactions to the drug. Table 3 and Table 4 herein provide safety data for pertuzumab. The safety data comprises any one or more (e.g. two, three, four or more) of the most common adverse events (AEs) or adverse reactions (ADRs) in Tables 3 and 4. For example, the safety data comprises information about neutropenia, febrile neutropenia, diarrhea and/or cardiac toxicity as disclosed herein.

“Efficacy data” refers to the data obtained in controlled clinical trial showing that a drug effectively treats a disease, such as cancer.

By “stable mixture” when referring to a mixture of two or more drugs, such as pertuzumab and trastuzumab” means that each of the drugs in the mixture essentially retains its physical and chemical stability in the mixture as evaluated by one or more analytical assays. Exemplary analytical assays for this purpose include: color, appearance and clarity (CAC), concentration and turbidity analysis, particulate analysis, size exclusion chromatography (SEC), ion-exchange chromatography (IEC), capillary zone electrophoresis (CZE), image capillary isoelectric focusing (iCIEF), and potency assay. In one embodiment, mixture has been shown to be stable for up to 24 hours at 5° C. or 30° C.

Administration “in combination” encompasses combined administration and separate administration, in which case, administration of one therapeutic agent can occur prior to, simultaneously, and/or following, administration of another therapeutic agents. Thus, administration of pertuzumab and trastuzumab in combination (or administration of a combination of pertuzumab and trastuzumab) encompasses combined administration and separate administration in either order.

A drug that is administered “concurrently” with one or more other drugs is administered during the same treatment cycle, on the same day of treatment as the one or more other drugs, and, optionally, at the same time as the one or more other drugs. For instance, for cancer therapies given every 3-weeks, the concurrently administered drugs are each administered on day-1 of a 3-week cycle.

### II. Antibody and Chemotherapy Compositions

The HER2 antigen to be used for production of antibodies may be, e.g., a soluble form of the extracellular domain of a HER2 receptor or a portion thereof, containing the desired epitope. Alternatively, cells expressing HER2 at their cell surface (e.g. NIH-3T3 cells transformed to overexpress HER2; or a carcinoma cell line such as SK-BR-3 cells, see Stancovski et al. PNAS (USA) 88:8691-8695 (1991)) can be used to generate antibodies. Other forms of HER2 receptor useful for generating antibodies will be apparent to those skilled in the art.

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

The anti-HER2 antibodies used in accordance with the present invention, trastuzumab and pertuzumab, are commercially available.

(i) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

U.S. Pat. No. 6,949,245 describes production of exemplary humanized HER2 antibodies which bind HER2 and block ligand activation of a HER receptor.

Humanized HER2 antibodies specifically include trastuzumab as described in Table 3 of U.S. Pat. No. 5,821,337 expressly incorporated herein by reference and as defined herein; and humanized 2C4 antibodies such as pertuzumab as described and defined herein.

The humanized antibodies herein may, for example, comprise nonhuman hypervariable region residues incorporated into a human variable heavy domain and may further comprise a framework region (FR) substitution at a position selected from the group consisting of 69H, 71H and 73H utilizing the variable domain numbering system set forth in Kabat et al., Sequences of Proteins of Immunological Interest, 5th Ed. Public Health Service, National Institutes of Health, Bethesda, Md. (1991). In one embodiment, the humanized antibody comprises FR substitutions at two or all of positions 69H, 71H and 73H.

An exemplary humanized antibody of interest herein comprises variable heavy domain complementarity determining residues GFTFTDYTMX (SEQ ID NO: 17), where X is preferably D or S; DVNPNSGGSIYNQRFKG (SEQ ID NO:18); and/or NLGPSFYFDY (SEQ ID NO:19), optionally comprising amino acid modifications of those CDR residues, e.g. where the modifications essentially maintain or improve affinity of the antibody. For example, an antibody variant for use in the methods of the present invention may have from about one to about seven or about five amino acid substitutions in the above variable heavy CDR sequences. Such antibody variants may be prepared by affinity maturation, e.g., as described below.

The humanized antibody may comprise variable light domain complementarity determining residues KASQDVSIGVA (SEQ ID NO:20); SASYX¹X²X³, where X¹ is preferably R or L, X² is preferably Y or E, and X³ is preferably T or S (SEQ ID NO:21); and/or QQYYIYPYT (SEQ ID NO:22), e.g. in addition to those variable heavy domain CDR residues in the preceding paragraph. Such humanized antibodies optionally comprise amino acid modifications of the above CDR residues, e.g. where the modifications essentially maintain or improve affinity of the antibody. For example, the antibody variant of interest may have from about one to about seven or about five amino acid substitutions in the above variable light CDR sequences. Such antibody variants may be prepared by affinity maturation, e.g., as described below.

The present application also contemplates affinity matured antibodies which bind HER2. The parent antibody may be a human antibody or a humanized antibody, e.g., one comprising the variable light and/or variable heavy sequences of SEQ ID Nos. 7 and 8, respectively (i.e. comprising the VL and/or VH of pertuzumab). An affinity matured variant of pertuzumab preferably binds to HER2 receptor with an affinity superior to that of murine 2C4 or pertuzumab (e.g. from about two or about four fold, to about 100 fold or about 1000 fold improved affinity, e.g. as assessed using a HER2-extracellular domain (ECD) ELISA). Exemplary variable heavy CDR residues for substitution include H28, H30, H34, H35, H64, H96, H99, or combinations of two or more (e.g. two, three, four, five, six, or seven of these residues). Examples of variable light CDR residues for alteration include L28, L50, L53, L56, L91, L92, L93, L94, L96, L97 or combinations of two or more (e.g. two to three, four, five or up to about ten of these residues).

Humanization of murine 4D5 antibody to generate humanized variants thereof, including trastuzumab, is described in U.S. Pat. Nos. 5,821,337, 6,054,297, 6,407,213, 6,639,055, 6,719,971, and 6,800,738, as well as Carter et al. PNAS (USA), 89:4285-4289 (1992). HuMAb4D5-8 (trastuzumab) bound HER2 antigen 3-fold more tightly than the mouse 4D5 antibody, and had secondary immune function (ADCC) which allowed for directed cytotoxic activity of the humanized antibody in the presence of human effector cells. HuMAb4D5-8 comprised variable light (V_{L}) CDR residues incorporated in a V_{L} κ subgroup I consensus framework, and variable heavy (V_{H}) CDR residues incorporated into a V_{H} subgroup III consensus framework. The antibody further comprised framework region (FR) substitutions as positions: 71, 73, 78, and 93 of the V_{H} (Kabat numbering of FR residues; and a FR substitution at position 66 of the V_{L} (Kabat numbering of FR residues). trastuzumab comprises non-A allotype human γ1 Fc region.

Various forms of the humanized antibody or affinity matured antibody are contemplated. For example, the humanized antibody or affinity matured antibody may be an antibody fragment. Alternatively, the humanized antibody or affinity matured antibody may be an intact antibody, such as an intact IgG1 antibody.

(ii) Pertuzumab Compositions

In one embodiment of a HER2 antibody composition, the composition comprises a mixture of a main species pertuzumab antibody and one or more variants thereof. The preferred embodiment herein of a pertuzumab main species antibody is one comprising the variable light and variable heavy amino acid sequences in SEQ ID Nos. 5 and 6, and most preferably comprising a light chain amino acid sequence of SEQ ID No. 11, and a heavy chain amino acid sequence of SEQ ID No. 12 (including deamidated and/or oxidized variants of those sequences). In one embodiment, the composition comprises a mixture of the main species pertuzumab antibody and an amino acid sequence variant thereof comprising an amino-terminal leader extension. Preferably, the amino-terminal leader extension is on a light chain of the antibody variant (e.g. on one or two light chains of the antibody variant). The main species HER2 antibody or the antibody variant may be an full length antibody or antibody fragment (e.g. Fab of F(ab′)2 fragments), but preferably both are full length antibodies. The antibody variant herein may comprise an amino-terminal leader extension on any one or more of the heavy or light chains thereof. Preferably, the amino-terminal leader extension is on one or two light chains of the antibody. The amino-terminal leader extension preferably comprises or consists of VHS-. Presence of the amino-terminal leader extension in the composition can be detected by various analytical techniques including, but not limited to, N-terminal sequence analysis, assay for charge heterogeneity (for instance, cation exchange chromatography or capillary zone electrophoresis), mass spectrometry, etc. The amount of the antibody variant in the composition generally ranges from an amount that constitutes the detection limit of any assay (preferably N-terminal sequence analysis) used to detect the variant to an amount less than the amount of the main species antibody. Generally, about 20% or less (e.g. from about 1% to about 15%, for instance from 5% to about 15%) of the antibody molecules in the composition comprise an amino-terminal leader extension. Such percentage amounts are preferably determined using quantitative N-terminal sequence analysis or cation exchange analysis (preferably using a high-resolution, weak cation-exchange column, such as a PROPAC WCX-10™ cation exchange column). Aside from the amino-terminal leader extension variant, further amino acid sequence alterations of the main species antibody and/or variant are contemplated, including but not limited to an antibody comprising a C-terminal lysine residue on one or both heavy chains thereof, a deamidated antibody variant, etc.

Moreover, the main species antibody or variant may further comprise glycosylation variations, non-limiting examples of which include antibody comprising a G1 or G2 oligosaccharide structure attached to the Fc region thereof, antibody comprising a carbohydrate moiety attached to a light chain thereof (e.g. one or two carbohydrate moieties, such as glucose or galactose, attached to one or two light chains of the antibody, for instance attached to one or more lysine residues), antibody comprising one or two non-glycosylated heavy chains, or antibody comprising a sialidated oligosaccharide attached to one or two heavy chains thereof etc.

The composition may be recovered from a genetically engineered cell line, e.g. a Chinese Hamster Ovary (CHO) cell line expressing the HER2 antibody, or may be prepared by peptide synthesis.

For more information regarding exemplary pertuzumab compositions, see U.S. Pat. Nos. 7,560,111 and 7,879,325 as well as US 2009/0202546A1.

(In) Trastuzumab Compositions

The trastuzumab composition generally comprises a mixture of a main species antibody (comprising light and heavy chain sequences of SEQ ID NOS: 13 and 14, respectively), and variant forms thereof, in particular acidic variants (including deamidated variants). Preferably, the amount of such acidic variants in the composition is less than about 25%, or less than about 20%, or less than about 15%. See, U.S. Pat. No. 6,339,142. See, also, Harris et al., J. Chromatography, B 752:233-245 (2001) concerning forms of trastuzumab resolvable by cation-exchange chromatography, including Peak A (Asn30 deamidated to Asp in both light chains); Peak B (Asn55 deamidated to isoAsp in one heavy chain); Peak 1 (Asn30 deamidated to Asp in one light chain); Peak 2 (Asn30 deamidated to Asp in one light chain, and Asp102 isomerized to isoAsp in one heavy chain); Peak 3 (main peak form, or main species antibody); Peak 4 (Asp102 isomerized to isoAsp in one heavy chain); and Peak C (Asp102 succinimide (Asu) in one heavy chain). Such variant forms and compositions are included in the invention herein.

### III. Selecting Patients for Therapy

Detection of HER2 expression or amplification can be used to select patients for treatment in accordance with the present invention. Several FDA-approved commercial assays are available to identify HER2-positive, HER2-expressing, HER2-overexpressing or HER2-amplified cancer patients. These methods include HERCEPTEST® (Dako) and PATHWAY® HER2 (immunohistochemistry (IHC) assays) and PathVysion® and HER2 FISH pharmDx™ (FISH assays). Users should refer to the package inserts of specific assay kits for information on the validation and performance of each assay.

For example, HER2 expression or overexpression may be analyzed by IHC, e.g. using the HERCEPTEST® (Dako). Paraffin embedded tissue sections from a tumor biopsy may be subjected to the IHC assay and accorded a HER2 protein staining intensity criteria as follows:

Score 0 no staining is observed or membrane staining is observed in less than 10% of tumor cells.

Score 1+ a faint/barely perceptible membrane staining is detected in more than 10% of the tumor cells. The cells are only stained in part of their membrane.

Score 2+ a weak to moderate complete membrane staining is observed in more than 10% of the tumor cells.

Score 3+ a moderate to strong complete membrane staining is observed in more than 10% of the tumor cells.

Those tumors with 0 or 1+ scores for HER2 overexpression assessment may be characterized as HER2-negative, whereas those tumors with 2+ or 3+ scores may be characterized as HER2-positive.

Tumors overexpressing HER2 may be rated by immunohistochemical scores corresponding to the number of copies of HER2 molecules expressed per cell, and can been determined biochemically:

0=0-10,000 copies/cell,

1+=at least about 200,000 copies/cell,

2+=at least about 500,000 copies/cell,

3+=at least about 2,000,000 copies/cell.

Overexpression of HER2 at the 3+ level, which leads to ligand-independent activation of the tyrosine kinase (Hudziak et al., Proc. Natl. Acad. Sci. USA, 84:7159-7163 (1987)), occurs in approximately 30% of breast cancers, and in these patients, relapse-free survival and overall survival are diminished (Slamon et al., Science, 244:707-712 (1989); Slamon et al., Science, 235:177-182 (1987)).

The presence of HER2 protein overexpression and gene amplification are highly correlated, therefore, alternatively, or additionally, the use of in situ hybridization (ISH), e.g. fluorescent in situ hybridization (FISH), assays to detect gene amplification may also be employed for selection of patients appropriate for treatment in accordance with the present invention. FISH assays such as the INFORM™ (sold by Ventana, Ariz.) or PathVysion® (Vysis, Ill.) may be carried out on formalin-fixed, paraffin-embedded tumor tissue to determine the extent (if any) of HER2 amplification in the tumor.

Most commonly, HER2-positive status is confirmed using archival paraffin-embedded tumor tissue, using any of the foregoing methods.

Preferably, HER2-positive patients having a 2+ or 3+ IHC score and/or who are FISH or ISH positive are selected for treatment in accordance with the present invention. Patients with 3+ IHC score and FISH/ISH positivity are particularly suitable for treatment in accordance with the present invention.

HER2 mutations associated with responsiveness to HER2-directed therapy have also been identified. Such mutations include, without limitation, insertions in exon 20 of HER2, deletions around amino acid residues 755-759 of HER2, any of the mutations G309A, G309E, S310F, D769H, D769Y, V777L, P780-Y781insGSP, V842I, R896C (Bose et al., Cancer Discov 2013; 3:1-14), as well as previously reported identical non-synonymous putative activating mutations (or indels) in COSMIC database found in two or more unique specimens.

See also U.S. Pat. No. 7,981,418 for alternative assays for screening patients for therapy with pertuzumab, and the Examples.

### IV. Pharmaceutical Formulations

Therapeutic formulations of the HER2 antibodies used in accordance with the present invention are prepared for storage by mixing an antibody having the desired degree of purity with optional pharmaceutically acceptable carriers, excipients or stabilizers (Remington's Pharmaceutical Sciences 16^{th} edition, Osol, A. Ed. (1980)), generally in the form of lyophilized formulations or aqueous solutions. Antibody crystals are also contemplated (see US Pat Appln 2002/0136719). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized antibody formulations are described in WO 97/04801, expressly incorporated herein by reference.

Lyophilized antibody formulations are described in U.S. Pat. Nos. 6,267,958, 6,685,940 and 6,821,515, expressly incorporated herein by reference. The preferred HERCEPTIN® (trastuzumab) formulation is a sterile, white to pale yellow preservative-free lyophilized powder for intravenous (IV) administration, comprising 440 mg trastuzumab, 400 mg .alphaα,α-trehalose dehydrate, 9.9 mg L-histidine-HCl, 6.4 mg L-histidine, and 1.8 mg polysorbate 20, USP. Reconstitution of 20 mL of bacteriostatic water for injection (BWFI), containing 1.1% benzyl alcohol as a preservative, yields a multi-dose solution containing 21 mg/mL trastuzumab, at pH of approximately 6.0. For further details, see the trastuzumab prescribing information.

The preferred pertuzumab formulation for therapeutic use comprises 30 mg/mL pertuzumab in 20 mM histidine acetate, 120 mM sucrose, 0.02% polysorbate 20, at pH 6.0. An alternate pertuzumab formulation comprises 25 mg/mL pertuzumab, 10 mM histidine-HCl buffer, 240 mM sucrose, 0.02% polysorbate 20, pH 6.0.

The formulation of the placebo used in the clinical trials described in the Examples is equivalent to pertuzumab, without the active agent.

The formulation herein may also contain more than one active compound as necessary for the particular indication being treated, preferably those with complementary activities that do not adversely affect each other. Various drugs which can be combined with the HER dimerization inhibitor are described in the Method Section below. Such molecules are suitably present in combination in amounts that are effective for the purpose intended.

The formulations to be used for in vivo administration must be sterile. This is readily accomplished by filtration through sterile filtration membranes.

### V. Treatment Methods

According to the present invention, pertuzumab and trastuzumab are administered according to applicable prescribing information.

Pertuzumab is typically administered every three weeks by intravenous infusion, starting with a first 840 mg infusion administered over 60 minutes, followed by a second and any subsequent intravenous infusions of 420 mg administered over 30 to 60 minutes. Further details of suitable administration schedules are given in the trastuzumab Prescribing Information and in the Examples.

Trastuzumab is typically administered every three weeks by intravenous infusion starting with a first 8 mg/kg loading dose over 90 minutes, followed by a second and any subsequent intravenous infusions 6 mg/kg maintenance doses administered over 30 to 60 minutes. Further details of suitable administration schedules are given in the trastuzumab Prescribing Information and in the Examples.

Pertuzumab and trastuzumab can be administered during the same visit, in either order.

### VI. Articles of Manufacture

In another embodiment of the invention, an article of manufacture containing materials useful for the treatment of a colorectal, biliary, urothelial, bladder, salivary, or lung cancer is provided. The article of manufacture comprises a vial with a fixed dose of the pertuzumab, wherein the fixed dose is approximately 420 mg, approximately 525 mg, approximately 840 mg, or approximately 1050 mg of pertuzumab, such as 420 mg or 840 mg of pertuzumab. The article of manufacture preferably further comprises a package insert. The package insert may provide instructions to administer the fixed dose to a patient with HER2-positive, HER2-amplified, or HER2-mutated colorectal, biliary, urothelial, bladder, salivary, or lung cancer, in combination with trastuzumab, as described and claimed herein. In certain embodiment, the package insert provides instructions for the treatment of colorectal cancer, biliary cancer, urothelial cancer, bladder cancer, salivary cancer, or lung cancer, such as advanced (locally advanced or metastatic) and/or treatment resistant colorectal, biliary, urothelial, bladder, salivary, or lung cancer.

In one embodiment, the article of manufacture comprises two vials, wherein a first vial contains a fixed dose of approximately 840 mg of pertuzumab, and a second vial contains a fixed dose of approximately 420 mg of pertuzumab.

In another embodiment, the article of manufacture of comprises two vials, wherein a first vial contains a fixed dose of approximately 1050 mg of pertuzumab, and a second vial contains a fixed dose of approximately 525 mg of pertuzumab.

In one embodiment of an article of manufacture herein comprises an intravenous (IV) bag containing a stable mixture of pertuzumab and trastuzumab suitable for administration to a cancer patient. Optionally, the mixture is in saline solution; for example comprising about 0.9% NaCl or about 0.45% NaCl. An exemplary IV bag is a polyolefin or polyvinyl chloride infusion bag, e.g. a 250 mL IV bag. According to one embodiment of the invention, the mixture includes about 420 mg or about 840 mg of pertuzumab and from about 200 mg to about 1000 mg of trastuzumab (e.g. from about 400 mg to about 900 mg of trastuzumab).

Optionally, the mixture in the IV bag is stable for up to 24 hours at 5° C. or 30° C. Stability of the mixture can be evaluated by one or more assays selected from the group consisting of: color, appearance and clarity (CAC), concentration and turbidity analysis, particulate analysis, size exclusion chromatography (SEC), ion-exchange chromatography (IEC), capillary zone electrophoresis (CZE), image capillary isoelectric focusing (iCIEF), and potency assay.

In another embodiment, the article of manufacture comprises a single-dose vial containing about 420 mg of pertuzumab.

### VII. Deposit of Biological Materials

The following hybridoma cell lines have been deposited with the American Type Culture Collection, 10801 University Boulevard, Manassas, Va. 20110-2209, USA (ATCC):

| Antibody Designation | ATCC No. | Deposit Date |
|---|---|---|
| 4D5 | ATCC CRL 10463 | May 24, 1990 |
| 2C4 | ATCC HB-12697 | Apr. 8, 1999 |

TABLE 1
TABLE OF SEQUENCES

| Description | SEQ ID NO | FIG. |
|---|---|---|
| HER2 domain I | 1 | 1 |
| HER2 domain II | 2 | 1 |
| HER2 domain III | 3 | 1 |
| HER2 domain IV | 4 | 1 |
| 2C4 variable light | 5 | 2A |
| 2C4 variable heavy | 6 | 2B |
| 574/pertuzumab variable light | 7 | 2A |
| 574/pertuzumab variable heavy | 8 | 2B |
| human V_{L} consensus framework | 9 | 2A |
| Human V_{H} consensus framework | 10 | 2B |
| pertuzumab light chain | 11 | 3A |
| pertuzumab heavy chain | 12 | 3B |
| trastuzumab light chain | 13 | 4A |
| trastuzumab heavy chain | 14 | 4B |
| Variant pertuzumab light chain | 15 | 5A |
| Variant pertuzumab heavy chain | 16 | 5B |
| GFTFTDYTMX | 17 | |
| DVNPNSGGSIYNQRFKG | 18 | |
| NLGPSFYFDY | 19 | |
| KASQDVSIGVA | 20 | |
| SASYX¹X²X³ | 21 | |
| QQYYIYPYT | 22 | |

Further details of the invention are illustrated by the following non-limiting Examples. The disclosures of all citations in the specification are expressly incorporated herein by reference.

A list of abbreviations and definition of terms, as used throughout the specification, including the Examples, is provided in the following table.

LIST OF ABBREVIATIONS AND DEFINITIONs OF TERMS

| Abbreviation | Definition |
|---|---|
| AE | adverse event |
| ALT (SGPT) | alanine aminotransferase |
| ALP | alkaline phosphatase |
| ANC | absolute neutrophil count |
| AST (SGOT) | aspartate aminotransferase |
| AUC | area under the concentration-time curve |
| BID | twice daily |
| BML | below measurable limit |
| BSA | body surface area |
| BUN | blood urea nitrogen |
| BWFI | bacteriostatic water for injection |
| CBC | complete blood count |
| CFR | Code of Federal Regulations |
| CHF | congestive heart failure |
| CI | confidence interval |
| CISH | chromogenic in situ hybridization |
| CLIA | Clinical Laboratory Improvement Amendments |
| Cₘₐₓ | maximum concentration observed |
| CMP | comprehensive metabolic profile |
| COSMIC | Catalogue of Somatic Mutations in Cancer |
| CR | complete response |
| CRF | Case Report Form |
| CRO | contract research organization |
| CT | computerized tomography |
| CTCAE | Common Terminology Criteria for Adverse Events |
| ctDNA | circulating tumor DNA |
| cuSCC | cutaneous squamous cell carcinoma |
| DCR | disease control rate |
| DILI | drug-induced liver injury |
| EC | Ethics Committee |
| eCRF | electronic Case Report Form |
| ECG | electrocardiogram |
| ECHO | echocardiogram |
| ECOG PS | Eastern Cooperative Oncology Group Performance Status |
| EDC | electronic data capture |
| EGFR | epidermal growth factor receptor |
| ERK | extracellular signal-regulated kinase |
| FDA | Food and Drug Administration |
| FISH | fluorescence in situ hybridization |
| FSH | follicle stimulating hormone |
| G-CSF | granulocyte colony-stimulating factor |
| GCP | Good Clinical Practice |
| GGT | gamma-glutamyl transferase |
| GI | gastrointestinal |
| hCG | human chorionic gonadotrophin |
| HER2 | human epidermal growth factor 2 |
| HFSA | Heart Failure Society of America |
| HIPAA | Health Insurance Portability and Accountability Act |
| IB | Investigator Brochure |
| ICH | International Conference on Harmonisation |
| IHC | immunohistochemistry |
| ILD | interstitial lung disease |
| IMP | investigational medicinal product |
| IND | Investigational New Drug (application) |
| INR | International Normalized Ratio |
| IRB | Institutional Review Board |
| IWRS | interactive web response system |
| KA | keratoacanthoma |
| LPLV | last patient, last visit |
| LVEF | left ventricular ejection fraction |
| MAPK | mitogen-activated protein kinase |
| MedDRA | Medical Dictionary for Regulatory Activities |
| MEK | MAPK/ERK kinase |
| MRI | magnetic resonance imaging |
| MUGA | multiple-gated acquisition (scan) |
| NCI | National Cancer Institute |
| NGS | next generation sequencing |
| NSCLC | non-small cell lung cancer |
| NYHA | New York Heart Association |
| ORR | overall response rate |
| OS | overall survival |
| 1-year OS | one-year survival rate |
| PCR | polymerase chain reaction |
| PD | progressive disease |
| PET | positron emission tomography (scan) |
| PFS | progression-free survival |
| PO | orally |
| PR | partial response |
| PTCH-1 | protein patched homolog-1 (gene) |
| RECIST | Response Evaluation Criteria in Solid Tumors |
| RT-PCR | real-time polymerase chain reaction |
| RVO | retinal vein occlusion |
| SAE | serious adverse event |
| SCRI | Sarah Cannon Research Institute |
| SD | stable disease |
| SMO | smoothened (gene) |
| Tₘₐₓ | time to maximum concentration |
| ULN | upper limit of normal |
| USPI | U.S. Package Insert |
| VAF | variant allele frequency |
| WCBP | woman of childbearing potential |

### Example 1

### Phase IIa Clinical Study Evaluating Trastuzumab Plus Pertuzumab for Patients with Cancers Characterized by HER2 Overexpression, Amplification, or HER2-Activating Mutation

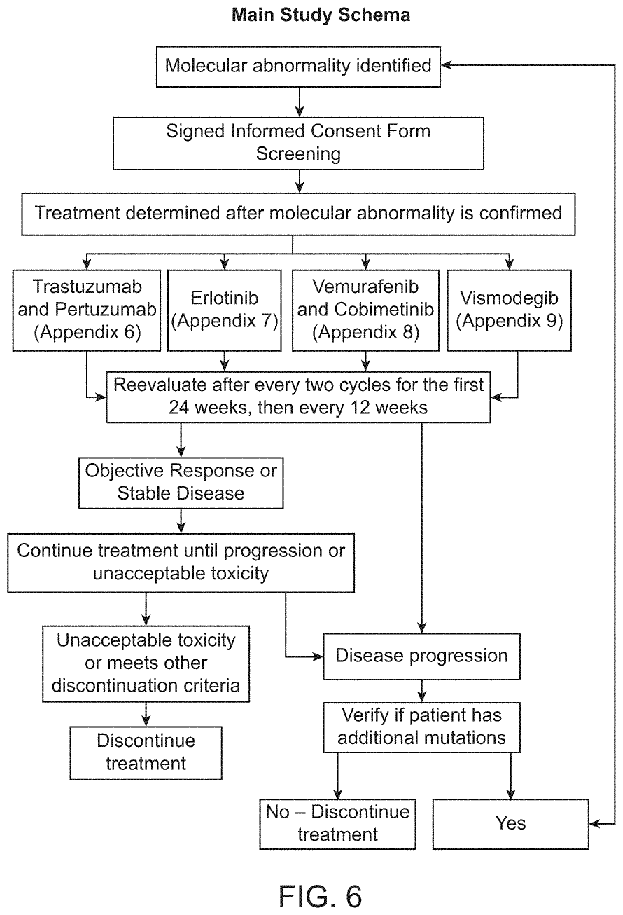

This is a multicenter, non-randomized, open-label Phase IIa study (MyPathwayStudy; ML28897; NCT2091141) conducted in the United States. Four different treatment regimens will be evaluated simultaneously in groups of patients who have advanced solid tumors that have progressed following administration of standard of care treatment, or for whom no standard therapy exists, or for whom therapies that will convey clinical benefit are not available and/or are not suitable options per the treating physician's judgment, and in whom a trial of targeted therapy is considered the best available treatment option. Treatment of patients with solid tumors that are characterized by HER2 overexpression, amplification, or HER2-activating mutation is one of the treatment regimens studied. The study schema for this clinical trial is shown in FIG. 6.

### Objectives

Primary Objective

The primary objective for this study is to evaluate the efficacy (determined by investigator-assessed overall response) of trastuzumab plus pertuzumab in patients with advanced solid tumors and: 1) with molecular alterations (mutations, gene expression abnormalities) predictive of response to one of these agents, 2) with no prior approved indication for use of these agents, 3) who are ineligible for an actively accruing Roche/Genentech-sponsored interventional trial, and 4) for whom therapies that will convey clinical benefit are not available and/or are not suitable options per the treating physician's judgment.

Secondary Objectives

The secondary objectives for this study are as follows:

To evaluate the safety and tolerability of the study medications for the tumor types studied

To collect and store molecular profiling data of all patients treated in this study, for the purpose of correlating treatment response with patterns of tumor genetic abnormalities

Exploratory Biomarker Objectives

The exploratory biomarker objectives for this study are as follows:

To evaluate the association of the levels and nature of somatic tumor specific mutations identified by blood-based next generation sequencing (NGS) and response to the study medications (i.e., predictive biomarkers), progression to a more severe disease state (i.e. prognostic biomarkers), acquired resistance to the study medications, evidence of activity of the study medications, and standard measures of efficacy.

To evaluate the association of levels and nature of somatic tumor-specific mutations identified by blood-based NGS to better understand the progression of disease.

Study Design

Inclusion Criteria

Patients must meet the following criteria for study entry:
Able to understand the nature of this trial and provide written informed consent
Age ≥18 years
Willing and able to comply with study and follow-up procedures
Life expectancy ≥12 weeks
Histologically documented metastatic cancer (solid tumors, not including hematologic malignancies)
Molecular testing results from Clinical Laboratory Improvement Amendments (CLIA)-certified laboratories showing that tumor tissue demonstrated at least one of the following abnormalities:
HER2 overexpression, amplification, or HER2-activating mutation
Molecular testing results used for patient eligibility must have been obtained from the most recent tumor biopsy. (Note: a new biopsy is not required.)
Patients who have received standard first-line therapy for metastatic cancer (except for the tumors for which no first-line therapy exists) and in whom a trial of targeted therapy is considered the best available treatment option. Eligible patients should not have available therapies that will convey clinical benefit and/or are not suitable options per the treating physician's judgment.
No previous treatment with the specific assigned study drug or any other drug sharing the same target
Progressive cancer at the time of study entry
Measurable or evaluable disease by Response Evaluation Criteria in Solid Tumors version 1.1 (RECIST v1.1)
Eastern Cooperative Oncology Group Performance Status (ECOG PS) score of 0, 1, or 2
Adequate hematologic function defined as:
Absolute neutrophil count ≥1000/μL
Hemoglobin ≥8 g/dL (may be achieved with erythropoietin agents or transfusions)
Platelets ≥75,000/μL
Adequate renal and liver function defined as:
Alanine aminotransferase and aspartate aminotransferase ≤2.5× the upper limit of normal (ULN) (≤5×ULN if considered due to primary or metastatic liver involvement)
Total bilirubin ≤1.5×ULN
Alkaline phosphatase <2×ULN (<5×ULN if considered due to tumor) Serum creatinine ≤2.0 mg/dL or calculated creatinine clearance ≥50 mL/min by Cockcroft-Gault formula
Females of childbearing potential, including females who have had tubal ligation, must have a negative serum pregnancy test <7 days prior to initial trial treatment.
Female patients of childbearing potential must agree to use acceptable methods of contraception.
Patients with solid tumors that have HER2 overexpression, amplification, or HER2-activating mutation as identified by assays performed at a Clinical Laboratory Improvement Amendments (CLIA)-certified laboratory.
Patients with breast, gastric, or gastroesophageal junction cancer must have HER2-activating mutation.
HER2 positivity as determined by protein overexpression using immunohistochemistry (IHC), by gene amplification using in situ hybridization (FISH or CISH), or tumors with HER2-activating kinase domain mutations identified by next generation sequencing (NGS) or real-time polymerase chain reaction (RT-PCR) will be accepted.
Assays using in situ hybridization (FISH or CISH) must indicate the presence of gene amplification with a HER2/CEP17 ratio of 2.0 or HER2 gene copy number >6.0.
Assays using IHC must indicate a score of 3+.
Assays using NGS of genes with known or potentially clinically relevant alterations or analysis by RT-PCR must identify clinically activating mutations (those with major coding disruptions resulting in an amino acid change that is likely to be detrimental to protein function, including premature stop codons or frameshift mutations early in the coding region).
In cases where multiple assays are done, HER2 positivity by any of the testing methodologies would make the patient eligible as long as eligibility criteria are fulfilled.
Left ventricular ejection fraction (LVEF) >50% or above the lower limit of the institutional normal range, whichever is lower

Exclusion Criteria

Patients who meet any of the following criteria will be excluded from study entry:
Patients with hematologic malignancies
Concurrent administration of any other anti-cancer therapy (except male patients with prostate cancer who are receiving androgen blockade): Bisphosphonates and denosumab are allowed.
Most recent anti-cancer therapy ≤28 days and have not recovered from the side effects, excluding alopecia
Radiation therapy within ≤14 days
Active or untreated brain metastases
Patients with treated brain metastases are eligible if they have minimal neurologic symptoms, evidence of stable disease (for at least 1 month) or response on follow-up scan, and require no corticosteroid therapy.
History of carcinomatous meningitis
Uncontrolled concurrent malignancy (early stage is allowed if not requiring active therapy or intervention)
Women who are breastfeeding
Any of the following cardiovascular events within 6 months prior to study entry: myocardial infarction, malignant hypertension, severe/unstable angina, symptomatic congestive heart failure, cerebral vascular accident, or transient ischemic attack
Pulmonary embolism within 30 days prior to study entry
History or presence of clinically significant ventricular or atrial dysrhythmia >Grade 2 (National Cancer Institute Common Terminology Criteria for Adverse Events version 4.0 [NCI CTCAE v4.0])
Patients with chronic, rate-controlled atrial arrhythmias who do not have other cardiac abnormalities are eligible.
Any other severe acute or chronic medical or psychiatric condition or laboratory abnormality that may increase the risk associated with study participation or may interfere with the interpretation of study results
Psychological, familial, sociological, or geographical conditions that do not permit compliance with the protocol
Eligible for another actively accruing Roche/Genentech-sponsored interventional clinical trial
Breast, gastric, or gastroesophageal junction cancer identified by HER2 amplification or overexpression
Previous treatment with any HER2-targeted therapy

Study Treatment

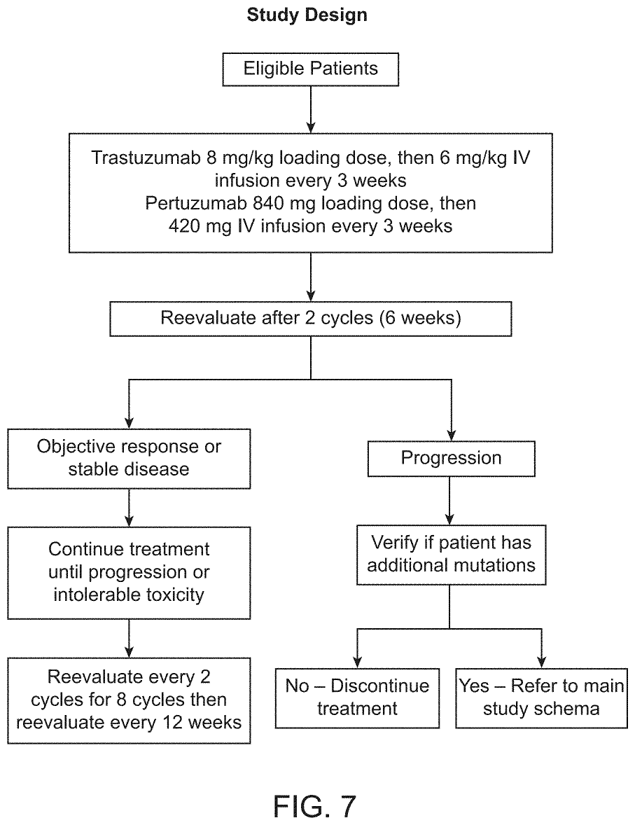

All patients will receive treatment with pertuzumab plus trastuzumab, given intravenously (IV) in cycles of 21 days (3 weeks) duration. A schema of the study design is presented in FIG. 7.

All patients will receive:
Trastuzumab 8 mg/kg intravenous (IV) loading dose, followed by 6 mg/kg, given by IV infusion every 3 weeks.
Pertuzumab 840 mg IV loading dose, followed by 420 mg, given by IV every 3 weeks.
The order of administration of trastuzumab and pertuzumab is according to investigator preference.
Both antibodies will be infused according to the U.S. Package Insert (USPI).
No routine premedications are required; however, patients who experience infusion-related symptoms may be premedicated as per standard institutional practice for subsequent infusions.

### Materials and Methods

Trastuzumab (Herceptin®)

Formulation

Trastuzumab is a sterile, white to pale yellow, preservative-free lyophilized powder for IV administration. Each vial of trastuzumab contains 440 mg of trastuzumab, 9.9 mg of L-histidine HCl, 6.4 mg of L-histidine, 440 mg of α,α-trehalose dihydrate, and 1.8 mg of polysorbate 20, USP. Reconstitution with 20 mL of the supplied bacteriostatic water for injection (BWFI) USP, containing 1.1% benzyl alcohol as a preservative, yields 21 mL of a multidose solution containing 21 mg/mL trastuzumab, at a pH of ˜6.

Dosage, Administration, and Storage

The 8 mg/kg loading dose of trastuzumab should be administered over 90 (±10) minutes. DO NOT ADMINISTER AS AN IV PUSH OR BOLUS. trastuzumab dosing will be based on the patient's baseline weight measurement. Weight will be measured on Day 1 of every 3-week treatment cycle. In case of a ≥10% change in weight, the trastuzumab dose should be re-calculated using the new weight. For the first infusion (Cycle 1), patients should be observed for 60 minutes from the end of the infusion for fever and chills, or other infusion-related reactions. If Cycle 1 is tolerated, then Cycle 2 and subsequent Q 21 day doses of 6 mg/kg of trastuzumab may be administered over 30 (±10) minutes, and patients will be observed as shown in Table 2. All infusion-related symptoms must have resolved before pertuzumab (if trastuzumab was given first) is given or the patient is discharged. Patients who experience infusion-related symptoms may be premedicated as per standard institutional practice for subsequent infusions.

With the exception of changes in trastuzumab dose due to a ≥10% change in weight from the baseline weight measurement, no changes in trastuzumab dosing are allowed at any time. trastuzumab will be held or discontinued in case of unacceptable toxicity.

Instructions for the administration of trastuzumab are listed below.

TABLE 2
Infusion Time and Post-Infusion Observation
Period for Trastuzumab

| | trastuzumab | Infusion | Post-Infusion |
|---|---|---|---|
| | Dose | Time | Observation Period |
| Infusion | (mg/kg) | (min)^{a} | (min)^{a} |
| 1^{st} infusion | 8 | 90 | 60 |
| 2^{nd} infusion | 6 | 30 | 30 |
| 3^{rd} and subsequent | 6 | 30 | None |
| infusions | | | |

^{a}After Cycle 1, ONLY shorten infusion and post-infusion observation times if the prior dose was well-tolerated.

Vials of trastuzumab are stable at 2° C.-8° C. (36° F.-46° F.) prior to reconstitution. Do not use beyond the expiration date stamped on the vial. A vial of trastuzumab reconstituted with BWFI, as supplied, is stable for 28 days after reconstitution when stored refrigerated at 2° C.-8° C. (36° F.-46° F.), and the solution is preserved for multiple uses. Discard any remaining multi-dose reconstituted solution after 28 days. If unpreserved sterile water for injections (not supplied) is used, the reconstituted trastuzumab solution should be used immediately and any unused portion must be discarded. DO NOT FREEZE

Trastuzumab that has been Reconstituted.

The solution of trastuzumab for infusion diluted in polyvinylchloride or polyethylene bags containing 0.9% sodium chloride for injection, USP, may be stored at 2° C.-8° C. (36° F.-46° F.) for up to 24 hours prior to use. Diluted trastuzumab has been shown to be stable for up to 24 hours at room temperature 15° C.-25° C.; however, since diluted trastuzumab contains no effective preservative the reconstituted and diluted solution should be stored refrigerated (2° C.-8° C.).

Dosage Modification

Toxicities will be evaluated utilizing the National Cancer Institute Common Terminology Criteria for Adverse Events version 4.0 (NCI CTCAE v4.0). If toxicity occurs, the toxicity will be graded, and appropriate supportive care treatment will be administered to decrease the signs and symptoms thereof.

Trastuzumab is well tolerated by most patients. If Grade 3-4 toxicity attributed to trastuzumab occurs, further dosing should be held until the toxicity improves to ≤Grade 1. trastuzumab should be restarted at full dose. If Grade 3-4 toxicity recurs, trastuzumab should be discontinued. Patients who are benefiting from therapy may continue treatment with pertuzumab, at the discretion of their treating physician.

Management of Toxicities

Management of specific trastuzumab-related toxicities is discussed below.

a. Hematologic Toxicity and Neutropenic Infections

In clinical trials, an increased incidence of anemia was observed in patients receiving trastuzumab plus chemotherapy compared with patients receiving chemotherapy alone. Most of these episodes of anemia were mild or moderate in intensity and were reversible. None of these events resulted in discontinuation of trastuzumab therapy.

In clinical trials, the incidences of moderate to severe neutropenia and febrile neutropenia were higher in patients receiving trastuzumab in combination with myelosuppressive chemotherapy as compared with those who received chemotherapy alone. In the post-marketing setting, deaths due to sepsis in patients with severe neutropenia have been reported in patients receiving trastuzumab and myelosuppressive chemotherapy. However, in controlled clinical trials (pre- and post-marketing), the incidence of septic deaths was not significantly increased. The pathophysiologic basis for exacerbation of neutropenia has not been determined. The effect of trastuzumab on the pharmacokinetics of chemotherapeutic agents has not been fully evaluated.

b. Management of Hematologic Toxicities with Trastuzumab

Care should be taken to carefully monitor the patient's hematologic status throughout the course of the trial. Use of hematopoietic growth factors to ameliorate hematologic toxicity is at the discretion of the physician investigator and should be in accordance with the American Society of Clinical Oncologists guidelines.

c. Trastuzumab Overdosage

There has been no instance of overdosage of trastuzumab in human clinical trials. Single doses of higher than 500 mg of trastuzumab have not been tested.

d. Cardiac Dysfunction

Signs and symptoms of cardiac dysfunction were observed in a number of women who received trastuzumab alone or in combination with chemotherapy, most often anthracycline-based treatment. Cardiac dysfunction was observed most frequently among patients who received trastuzumab plus adriamycin/cyclophosphamide chemotherapy (28%), compared with those who received adriamycin/cyclophosphamide alone (7%), trastuzumab plus paclitaxel (11%), paclitaxel alone (1%), or trastuzumab alone (7%). Severe disability or fatal outcome due to cardiac dysfunction was observed in approximately 1% of all patients.

In contrast to the irreversible nature of anthracycline-induced cardiomyopathy, the signs and symptoms of trastuzumab-induced cardiac dysfunction usually responded to treatment. Complete and partial responses were observed among patients with cardiac dysfunction. The risk appears to be independent of tumor response to therapy. Analysis of the clinical database for predictors of cardiac dysfunction revealed only advanced age and exposure to an anthracycline as possible risk factors. In the clinical trials, most patients with cardiac dysfunction responded to appropriate medical therapy, often including discontinuation of trastuzumab. In many cases, patients were able to resume treatment with trastuzumab. In a subsequent study using weekly paclitaxel and trastuzumab as first-line treatment for metastatic breast cancer, the observed incidence of serious cardiac dysfunction was 3% (N=95) (Seidman et al. 2001). Since the occurrence of cardiac dysfunction in the trastuzumab plus chemotherapy trial was an unexpected observation, no information is available regarding the most appropriate method for monitoring cardiac function in patients receiving trastuzumab.

Significant advances in the understanding and treatment of congestive heart failure (CHF) have been made in the past several years, with several new drugs demonstrating the ability to improve cardiac function. Patients who develop symptoms of CHF while on trastuzumab should be treated according to the Heart Failure Society of America (HFSA) guidelines (HFSA2010).

Since pertuzumab is also associated with a risk for cardiac dysfunction, the management of cardiac safety for patients receiving both drugs in the trial, as outlined in the next section, applies to both drugs.

e. Management of Cardiac Safety.

All patients must have a baseline evaluation of cardiac function including a measurement of LVEF by either a multiple-gated acquisition (MUGA) scan or an echocardiogram (ECHO) prior to entry into the study. Only patients with normal LVEF should be entered into this study. While receiving treatment, all patients will have regular monitoring of LVEF with MUGA or ECHO (every 12 weeks or as clinically indicated).

During the course of therapy with trastuzumab and pertuzumab patients should be monitored for signs and symptoms of heart failure (i.e., dyspnea, tachycardia, new unexplained cough, neck vein distention, cardiomegaly, hepatomegaly, paroxysmal nocturnal dyspnea, orthopnea, peripheral edema, and rapid unexplained weight gain). The diagnosis must be confirmed using the same method used to measure LVEF at baseline (either ECHO or MUGA).

f. Management of Symptomatic Cardiac Changes.

Patients who develop signs and symptoms of heart failure NCI CTCAE v4.0 Grade 2, 3, or 4 should have trastuzumab and pertuzumab held and should receive treatment for heart failure as prescribed by the HFSA (e.g., ACE inhibitors, angiotensin-II receptor blockers, β-blockers, diuretics, and cardiac glycosides, as needed; HFSA 2010). Consideration should be given to obtaining a cardiac consultation. LVEF should be reassessed after 3 weeks (using the same method of measurement).

If the symptoms of heart failure resolve with treatment, and cardiac function (as measured by ECHO or MUGA) improves, trastuzumab and pertuzumab may be restarted after discussion with the patient concerning the risks and benefits of continued therapy. If the patient is benefiting clinically from HER2-targeted treatment, the benefit of continued treatment may outweigh the risk of cardiac dysfunction. If trastuzumab and pertuzumab are restarted, continued surveillance with noninvasive measures of LVEF (MUGA or ECHO) will continue per protocol.

g. Management of Asymptomatic Decreases in LVEF

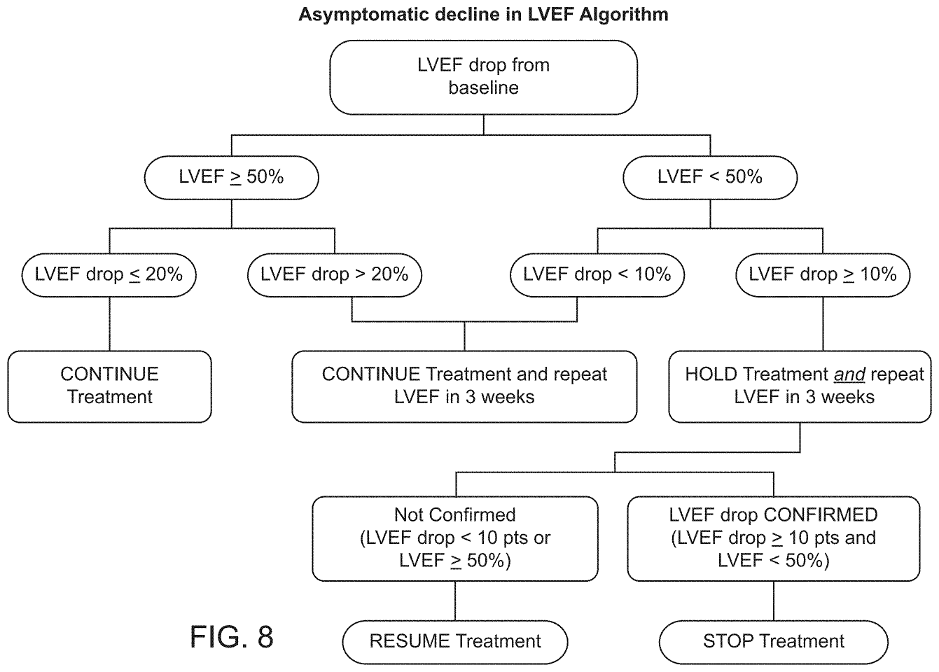

If routine LVEF measurements document asymptomatic LVEF decreases during treatment, patient management should follow guidelines outlined in FIG. 8.

Warnings and Precautions

a. Infusion Reactions to Trastuzumab

During the first infusion with trastuzumab, a symptom complex consisting of chills and/or fever is observed in approximately 40% of patients. Other signs and/or symptoms may include nausea, vomiting, pain, rigors, headache, cough, dizziness, rash, and asthenia. These symptoms are usually mild to moderate in severity, and occur infrequently with subsequent trastuzumab infusions. These symptoms may be treated as per standard institutional practice.

b. Serious Infusion-Associated Events with trastuzumab

Serious adverse reactions to trastuzumab infusion, including dyspnea, hypotension, wheezing, bronchospasm, tachycardia, reduced oxygen saturation, and respiratory distress, can be serious and/or potentially fatal. Most of these events have occurred either during or shortly after the start of the first trastuzumab infusion. Severe or moderate infusion-related symptoms may be managed by slowing or stopping the trastuzumab infusion, and implementing supportive therapy with oxygen, beta agonists, antihistamines, or corticosteroids.

If Grade 3 or Grade 4 toxicity occurs during the post-infusion observation period, the patient must be evaluated for a minimum of 1 hour from the time the toxicity was first observed until the resolution of any severe symptoms.

Patients who have an infusion-associated adverse event with trastuzumab should receive prophylactic treatment with antihistamines and/or corticosteroids before all subsequent trastuzumab infusions. Please refer to the Herceptin® USPI for specific prophylactic pre-medications that are recommended.

c. Other trastuzumab-Related Toxicity

In addition to infusion-related toxicity, some patients have reported abdominal pain, indigestion, diarrhea, nausea, vomiting, loss of appetite and dehydration. Allergic reactions have also been reported. One patient in a large research study developed antibodies to trastuzumab.

Pertuzumab (Perjeta®)

Formulation Pertuzumab is provided as a single-use formulation containing 30 mg/mL pertuzumab formulated in 20 mM L-histidine (pH 6.0), 120 mM sucrose, and 0.02% polysorbate 20. Each 20-cc vial contains approximately 420 mg of pertuzumab (14.0 mL/vial).

Dosage, Administration, and Storage

Withdraw the indicated volume of pertuzumab from the vial and add to a 250-cc IV bag of 0.9% sodium chloride injection. Gently invert the bag to the mix solution. DO NOT SHAKE VIGOROUSLY. Visually inspect the solution for particulates and discoloration prior to administration. The entire volume within the bag should be administered as a continuous IV infusion. The volume contained in the administration tubing should be completely flushed using a 0.9% sodium chloride injection.

The solution of pertuzumab for infusion diluted in polyethylene or non-PVC polyolefin bags containing 0.9% sodium chloride injection may be stored at 2° C.-8° C. (36° F.-46° F.) for up to 24 hours prior to use. Diluted pertuzumab has been shown to be stable for up to 24 hours at room temperature (2° C.-25° C.). However, since pertuzumab contains no preservative, the aseptically diluted solution should be stored refrigerated (2° C.-8° C.) for no more than 24 hours.

A rate-regulating device may be used for all pertuzumab infusions. When the study drug IV bag is empty, 50 mL of 0.9% sodium chloride injection may be added to the IV bag or an additional bag will be hung, and the infusion may be continued for a volume equal to that of the tubing to ensure complete delivery of pertuzumab.

Administration of pertuzumab should be performed in a setting with emergency equipment and staff who are trained to monitor medical situations and respond to medical emergencies. The initial dose of pertuzumab will be administered over 60 minutes and patients will be monitored for a further 60 minutes following the completion of the infusion for any adverse effects. The infusion should be slowed or interrupted if the patient experiences infusion-related symptoms. If infusion-related symptoms occur, patients will be monitored until complete resolution of signs and symptoms. If the infusion is well tolerated, subsequent doses may be administered over 30 minutes, and patients will be observed for a further 30 minutes for infusion-related symptoms as shown in Table 3 below.

All infusion-related symptoms must have resolved before the patient is discharged. Patients who experience infusion-associated symptoms may subsequently be premedicated as per standard institutional practice.

TABLE 3
Infusion Time and Post-Infusion
Observation Period for Pertuzumab

| | pertuzumab | Infusion | Post-Infusion |
|---|---|---|---|
| | Dose | Time | Observation Period |
| Infusion | (mg) | (min)^{a} | (min)^{a} |
| 1^{st} infusion | 840 | 60 | 60 |
| 2^{nd} infusion and | 420 | 30 | 30 |
| subsequent infusions | | | |

After Cycle 1, ONLY shorten infusion and post infusion observation times if the prior dose was well tolerated.

Infusion should be stopped in patients who develop dyspnea or clinically significant hypotension (defined per investigator discretion). Patients who experience an NCI CTCAE Grade 3 or 4 allergic reaction or acute respiratory distress syndrome should not receive additional pertuzumab.

Should extravasation of the study drug during the infusion, the following steps should be taken:

Discontinue the infusion.

Treat the extravasation according to institutional guidelines for extravasation of a non-caustic agent.

If a significant volume of the study drug infusion remains, restart the infusion at a more proximal site in the same limb or on the other side.

Storage: Vials of pertuzumab must be placed in a refrigerator 2° C.-8° C. (36° F.-46° F.) immediately upon receipt to ensure optional retention of physical and biochemical integrity and should remain refrigerated until immediately prior to use. DO NOT FREEZE and DO NOT SHAKE the pertuzumab vial. Protect from light.

### Dosage Modification

Pertuzumab is well tolerated by most patients. If Grade 3-4 toxicity attributed to pertuzumab occurs, further dosing should be held until the toxicity improves to Grade 1.

Pertuzumab should be restarted at full dose. If Grade 3-4 toxicity recurs, pertuzumab should be discontinued. Patients who are benefiting from therapy may continue treatment with trastuzumab, at the discretion of their treating physician. Management of specific pertuzumab-related toxicities is discussed below.

Pertuzumab Warnings and Precautions

a. Infusion-Associated Reactions

An infusion reaction was defined in the randomized trial for metastatic breast cancer as any event described as hypersensitivity, anaphylactic reaction, acute infusion reaction, or cytokine release syndrome occurring during an infusion or on the same day as the infusion. The initial dose of pertuzumab was given the day before trastuzumab and docetaxel to allow for the examination of pertuzumab-associated reactions. On the first day, when only pertuzumab was administered, the overall frequency of infusion reactions was 13.0% in the pertuzumab-treated group and 9.8% in the placebo-treated group. Less than 1% were Grade 3 or 4. The most common infusion reactions 1.0%) were pyrexia, chills, fatigue, headache, asthenia, hypersensitivity, and vomiting.

During the second cycle when all drugs were administered on the same day, the most common infusion reactions in the pertuzumab-treated group 1.0%) were fatigue, dysgeusia, hypersensitivity, myalgia, and vomiting.

In the randomized trial, the overall frequency of hypersensitivity/anaphylaxis reactions was 10.8% in the pertuzumab-treated group and 9.1% in the placebo-treated group. The incidence of Grade 3-4 hypersensitivity/anaphylaxis reactions was 2% in the pertuzumab-treated group and 2.5% in the placebo-treated group according to NCI CTCAE v3.0. Overall, 4 patients in pertuzumab-treated group and 2 patients in the placebo-treated group experienced anaphylaxis.

Observe patients closely for 60 minutes after the first infusion and for 30 minutes after subsequent infusions of pertuzumab. If a significant infusion-associated reaction occurs, slow or interrupt the infusion and administer appropriate medical therapies as per standard institutional practice. Monitor patients carefully until complete resolution of signs and symptoms. Consider permanent discontinuation in patients with severe infusion reactions.

b. Risk of Cardiotoxicity

Pertuzumab is directed at the HER2 receptor and is associated with a risk of cardiac dysfunction. In pertuzumab single-agent Phase II studies, a fall in LVEF of 10% to a LVEF value of <50% was observed in 7% of patients who had a post-baseline LVEF assessment. Nine of these patients had received prior anthracycline treatment. Overall, three symptomatic cardiac failure events have been reported in approximately 550 patients treated with pertuzumab across all studies. Two of these cases occurred in patients with metastatic breast cancer who had received prior anthracyclines.

Patients with significant cardiac disease or baseline LVEF below 50% are not eligible for this study. Risk factors for pertuzumab-associated cardiac dysfunction are not known at this time. The risk of cardiac dysfunction should be carefully weighed against the potential benefit in patients who have received prior anthracyclines.

Since pertuzumab and trastuzumab have overlapping potential cardiac toxicity, the management of cardiotoxicity in this study arm should consider both treatments.

c. Embryo-Fetal Toxicity (for Trastuzumab or Pertuzumab)

There are no clinical studies of trastuzumab or pertuzumab in pregnant women. Immunoglobulin G1 (IgG1) is known to cross the placental barrier. Studies in animals have resulted in oligohydramnios, delayed renal development, and death.

It is not known whether trastuzumab or pertuzumab is excreted in breast milk. As maternal IgG1 is excreted in milk and either monoclonal antibody could harm infant growth and development, women should be advised to discontinue nursing during pertuzumab or trastuzumab therapy and not to breastfeed for at least 7 months following the last dose of either monoclonal antibody.

d. Follow-Up of Pregnancies

Infants born to female patients or female partners of male patients exposed to trastuzumab/pertuzumab must be followed for 1 year after birth. Additional information will be requested by the Sponsor at specific time points during and after the pregnancy (i.e., at the end of the second trimester, 2 weeks after expected date of delivery, and 3, 6, and 12 months of the infant's life).

e. Most Common Adverse Reactions

The most common adverse reactions (>30%) seen with pertuzumab in combination with trastuzumab and docetaxel were diarrhea, alopecia, neutropenia, nausea, fatigue, rash, and peripheral neuropathy. The most common NCI CTCAE (v 3.0) Grade 3-4 adverse reactions (>2%) were neutropenia, febrile neutropenia, leukopenia, diarrhea, peripheral neuropathy, anemia, asthenia, and fatigue.

Efficacy results of treatment with pertuzumab+trastuzumab of patients with various HER2 amplifying/overexpressing cancer types are shown in the following Table 4.

TABLE 4
Efficacy of Treatment with trastuzumab plus pertuzumab in
Patients with HER2 Amplification/Overexpression (N = 114*)

| | Number of | Response, n (%) | | | | | | | |
|---|---|---|---|---|---|---|---|---|---|
| Primary Site | Patients | CR | | PR | | SD > 120 days | | ORR (95% CI) | |
| Colorectal | 37 | 0 | | 14 | (38) | 4 | (11) | 38 | (23, 55) |
| Lung, non-small-cell | 16 | 0 | | 2 | (13) | 2 | (13) | 13 | (2, 38) |
| Bladder | 9 | 1 | (11) | 2 | (22) | 2 | (22) | 33 | (8, 70) |
| Pancreas | 9 | 0 | | 2 | (22) | 1 | (11) | 22 | (3, 60) |
| Biliary | 7 | 0 | | 2 | (29) | 3 | (38) | 29 | (4, 71) |
| Ovary | 8 | 0 | | 1 | (13) | 0 | | 13 | (0, 53) |
| Uterus | 7 | 0 | | 0 | | 0 | | 0 | |
| Salivary gland | 5 | 1 | (20) | 3 | (60) | 0 | | 80 | (28, >99) |
| Other (11 sites)† | 16 | 1 | (6) | 1 | (6) | 3 | (19) | 13 | (2, 38) |
| TOTAL | 114 | 3 | (3) | 27 | (24) | 16 | (14) | 26 | (19, 35) |

*Includes 12 patients with amplification/overexpression + mutation.
†Responses occurred in patients with adenocarcinomas of the prostate (1) and skin (apocrine) (1).
CR, complete response; ORR, objective response rate; PR, partial response; SD, stable disease; CI, confidence interval.

Further results are described in the following Examples.

### Example 2

### Pertuzumab+Trastuzumab for Treatment of Patients with Metastatic Colorectal Cancer (mCRC)

Colorectal cancer is the third leading cause of cancer deaths in the United States. Colorectal patients have a poor prognosis, with 5-year survival rates of 12.5% (Siegel R. et al., CA Cancer J Clin. 2014, 64:104-17). Among recent advances in precision medicine, HER2 has emerged as a potential therapeutic target for advanced colon cancer, however, no HER2-targeted therapies are currently approved for metastatic colorectal cancer (mCRC).

Study Design/Treatment

Eligible patients in this analysis had treatment refractory HER2-amplified/overexpressed mCRC, as assessed by next-generation sequencing (NGS), fluorescent or chromogenic in situ hybridization (FISH or CISH; signal ratio >2.0 or copy number <6), and/or immunohistochemistry (IHC; 3+), per local institutional standards. Patients with active brain metastases, concurrent active anti-cancer therapy, pregnancy, or contraindications to pertuzumab or trastuzumab were excluded. Patients received standard doses of pertuzumab+trastuzumab (pertuzumab: 840 mg intravenous [IV] loading dose, followed by 420 mg IV every 3 weeks; trastuzumab: 8 mg/kg IV loading dose, followed by 6 mg/kg IV every 3 weeks) until disease progression or unacceptable toxicity. The primary endpoint is investigator-assessed objective response rate (ORR).

Assessments and Statistical Methods

Tumor response was evaluated by the investigator every 6 weeks for the first 24 weeks and every 12 weeks after. Response was assessed by RECIST v1.1. For further details see Example 1.

Results

By the cut-off time, 34 patients with treatment-refractory HER2-amplified/overexpressed metastatic colorectal cancer (mCRC) enrolled in the MyPathway (NCT2091141) multicenter, open-label, phase IIA study described in Example 1, were treated as described above.

TABLE 5
Baseline demographics and clinical characteristics
of patients with HER2-amplified/overexpressed mCRC

| | Patients^{a} (n = 34) | |
|---|---|---|
| Median age, y (range) | 57 | (25-77) |
| Female, n (%) | 19 | (55.9) |
| Male, n (%) | 15 | (44.1) |
| Tumor site, n (%) | | |
| Colon | 23 | (67.6) |
| Right side | 8 | (23.5) |
| Left side | 14 | (41.1) |
| Transverse | 1 | (2.9) |
| Rectum | 11 | (32.4) |
| HER2 testing method, n (%)^{b} | | |
| NGS | 32 | (94.1) |
| FISH/CISH | 11 | (32.4) |
| IHC 3+ | 7 | (20.6) |
| Median number of prior regimens (range) | 4 | (1-9) |
| KRAS status, n (%) | | |
| Wild-type | 25 | (73.5) |
| Mutated | 9 | (26.5) |
| Prior anti-EGFR therapy, n (%)c, d | 20 | (80.0) |
| Cetuximab + chemotherapy^{e} | 14 | (56) |
| Panitumumab + chemotherapy^{f} | 10 | (40.0) |
| None | 5 | (20.0) |

^{a}One patient also had mutated HER2.
^{b}Some patients had multiple test types.
cPercentages are calculated based on patients with wild-type KRAS.
d Patients may have received more than one line of anti-EGFR therapy.
^{e}One patient in this group also received cetuximab monotherapy in a different treatment line.
^{f}Three patients in this group also received panitumumab monotherapy in a different treatment line.

Treatment Exposure and Clinical Outcomes

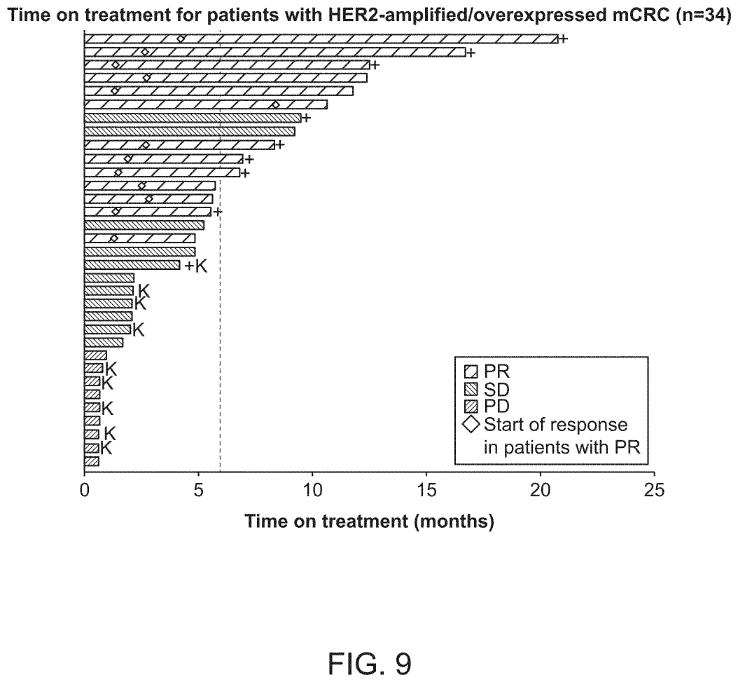

Median follow up was 5.6 (range 1.2-22.1) months. The median time on treatment was 4.1 (range 0-20.7) months. The time on treatment by patient is shown in FIG. 9.

ORR was 38.2% (n=13, 95% confidence interval [CI]; 22.2-56.4) and CBR was 50.0% (n=17; 95% CI, 32.4-64.6). All 13 responders, 7 with ongoing treatment, achieved PR as their best response. The median duration of response was 10.3 (range, 1.4-15.7) months. This group includes a patient with a concomitant HER2 mutation (S310F).

Four (11.8%) patients, one with ongoing treatment, had SD for greater than 4 months.

Seven (20.5%) patients, one with ongoing treatment, had SD for less than or equal to 4 months. This group includes one patient with a concomitant EGFR alteration.

Ten (29.4%) patients had progressive disease (PD).

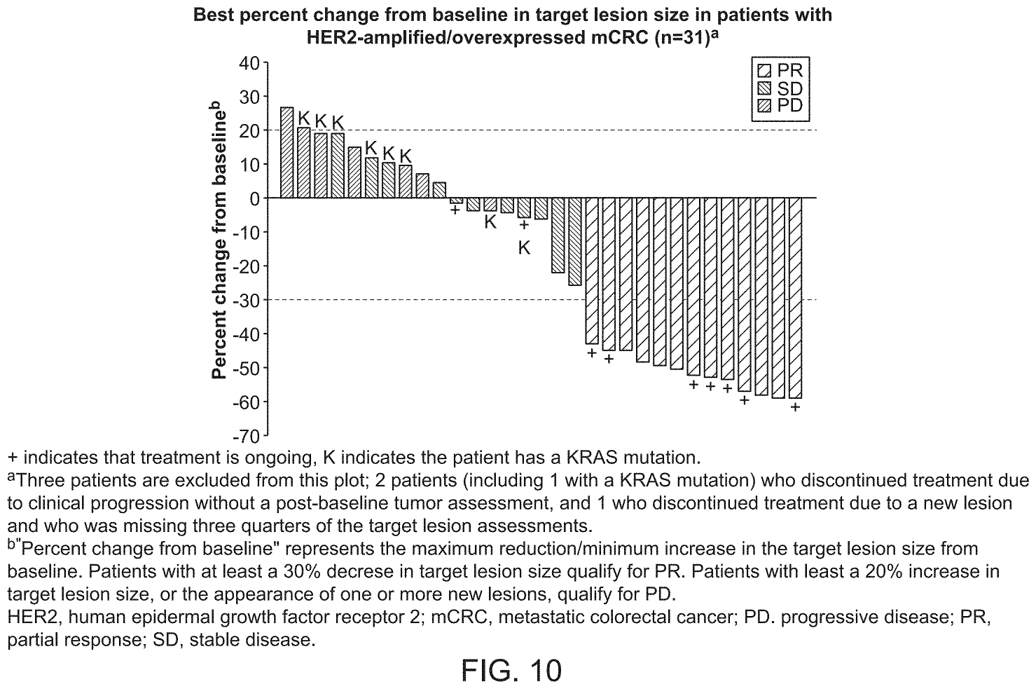

The best percentage from baseline in the target lesion size by patient in shown in FIG. 10.

TABLE 6
Outcomes by clinical characteristics in patients with HER2-amplified or overexpressed mCRC

| | | | | | Median | | | | | |
|---|---|---|---|---|---|---|---|---|---|---|
| | | | | | duration of | | | | | |
| Clinical | ORR, n | | CBR, n | | clinical benefit, | | Median PF S, | | Median OS, | |
| characteristic | (% [95% CI]) | | (% [95% CI]) | | months (95% CI) | | months (95% CI) | | months (95% CI) | |
| All patients | 13 | (38.2 [22.2-56.4]) | 17 | (50 [32.4-67.6]) | 10.3 | (4.3-NE) | 4.6 | (1.6-9.8) | 10.3 | (7.2-22.1) |
| (n = 34) | | | | | | | | | | |
| KRAS status | | | | | | | | | | |
| Wild-type (n = 25) | 13 | (52.0 [31.3-72.2 | 17 | (68.0 [46.5-85.1]) | 10.3 | (4.3-NE) | 5.7 | (3.6-12.4) | 14.0 | (8.0-22.1) |
| Mutated (n = 9) | 0 | (0 [NE-NE]) | 0 | (0 [NE-NE]) | NA | | 1.4 | (1.1-2.8) | 5.0 | (1.2-103.) |
| Number of prior | | | | | | | | | | |
| regimens | | | | | | | | | | |
| >4 (n = 12) | 4 | (33.3 [9.9-65.1]) | 4 | (33.3 [9.9-65.1]) | 2.8 | (2.8-NE) | 2.2 | (1.3-5.6) | 8.0 | (1.8-NE) |
| ≥4 (n = 12) | 9 | (40.9 [20.7-63.6]) | 13 | ((59.1 [36.4-79.3]) | 10.3 | (4.3-NE) | 5.6 | (2.7-12.4) | 10.3 | (7.2-22.1) |
| Tumor site | | | | | | | | | | |
| Colon, left side | 6 | (42.9 [17.1-71.1]) | 9 | (64.3 [35.1-87.2]) | 10.4 | (9.8-11.1) | 9.8 | (1.4-12.4) | 11.5 | (8.5-22.1) |
| (n = 14) | 1 | (12.5 [0.3-52.7]) | | | 10.3 | (NE-NE) | 1.4 | (1.1-3.9) | | |
| Colon, right side | 5 | (45.5 [16.7-76.6]) | 1 | (12.5 [0.3-52.7]) | 5.0 | (2.8-NE) | 5.6 | (1.3-11.1) | 4.5 | (1.2-14.0) |
| (n = 8) | | | | | | | | | | |
| Rectum (n = 11) | | | 6 | (54.5 [23.4-83.3]) | | | | | 10.3 | (1.8-NE) |

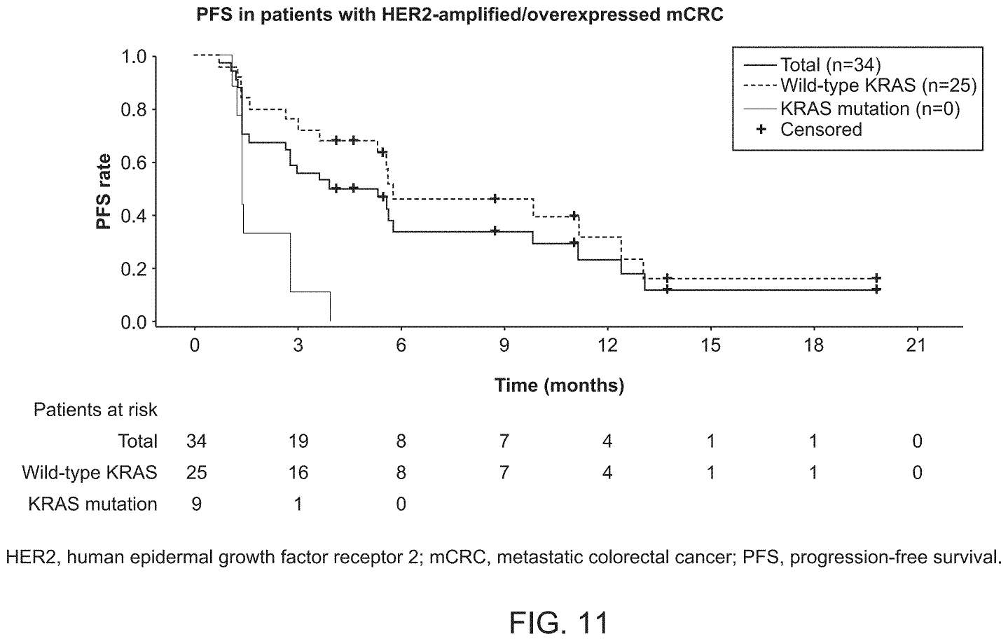

By data cut-off 73.5% (n=25) of patients had experienced a PFS event (tumor progression [n=23] or death [n=2]). Median PFS was 4.6 (95% CI, 1.6-9.8 months) as shown in Table 6 and FIG. 11. Patients with wild-type KRAS had a higher median PFS than patients with mutated KRAS (5.7 [95% CI, 3.5-12.4] months vs. 1.4 [95% CI, 1.1-2.8] months, respectively).

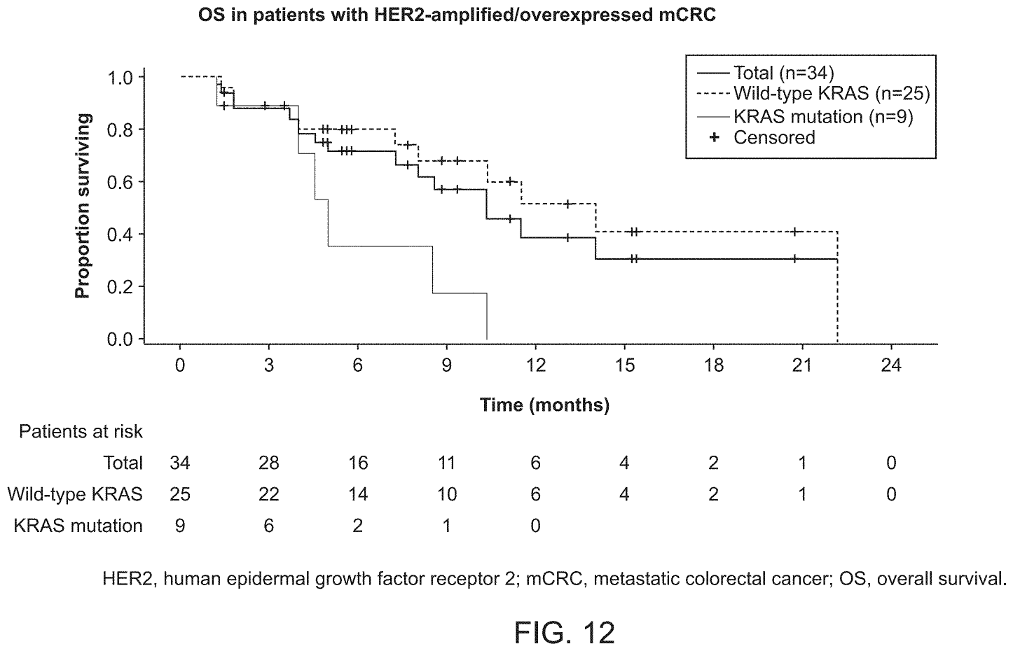

By data cut-off 50.0% of patients (n=17) had died. Thirteen patients died due to disease progression, 1 died from suspected brain metastases, and 3 died from unknown or unspecified causes. Median OS was 10.3 (05% CI, 7.2-22.1) months as shown in Table 6 and FIG. 12. Patients with wild-type KRAS had a higher median OS than patients with mutates KRAS (14.0 [95% CI, 8.0-22.1] months vs. 5.0 [95% CI, 1.2-10.3] months, respectively).

The safety profiles were consistent with the product labels for pertuzumab and trastuzumab.

### Conclusions

These data suggest that dual HER2-targeted therapy with pertuzumab+trastuzumab, a chemotherapy-free regimen, is active in patients with heavily pre-treated, HER2-amplified/overexpressed mCRC. ORR was 38.2%, with durable responses (median 10.3 months), and CBR was 50.0%. pertuzumab+trastuzumab treatment appeared to have higher activity in patients with wild-type KRAS tumors (ORR 52%, CBR 68%) compared with KRAS-mutated cohort (ORR 0%, CBR 0%). An analysis of 3256 patients with CRC indicated that HER2 amplification/overexpression is associated with KRAS wild-type tumor status (Richman S D et al., J Pathol 2016, 238:562-70). While ORR was lower in patients with right-sided colon cancer (12.5%) compared with left-sided colon (42.9%) or rectal cancer (45.5%), a higher percentage of right-sided colon tumors had mutated KRAS in this analysis (62.5% vs. 27.3%, respectively).

### Example 3

### Pertuzumab+Trastuzumab for Treatment of Patients with Metastatic Biliary Cancer

Biliary cancers have a high mortality rate, with limited treatment options. While HER2 is overexpressed in 9-20% of biliary cancers, it has not been fully explored as a therapeutic target.

11 patients with HER2-positive refractory metastatic biliary cancer with HER2 amplification/overexpression or putative activating mutations by gene sequencing, FISH, or IHC (HER2-amplified/overexpressed, n=8; HER2-mutated, n=3 [D277Y/D297Y, S310F, and A775-G776insYVMA]) enrolled in the MyPathway (NCR02091141) open-label, multicenter, phase IIA study described in Example 1 received standard doses of pertuzumab+trastuzumab until disease progression or unacceptable toxicity. The primary endpoint is investigator-assessed overall response rate (RECIST v1.1).

At a median follow-up of 4.2 (range 2.0-12.0) months, 4 patients had partial responses (PR) and 3 had stable disease (SD) for >4 months (Table 6). Safety was consistent with the package inserts. The results are summarized in Table 7.

TABLE 7

| | |
|---|---|
| HER2- | |
| amplified/ | |
| overexpressed | HER2-mutated |
| (n = 8) | (n = 3) |

| | | | | |
|---|---|---|---|---|
| Median age, y (range) | 61 | (45-77) | 57 | (56-67) |
| Female | 5 | (62.5) | 1 | (33.3) |

ECOG score, n (%)

| 0 | 3 | (37.5) | 1 | (33.3) |
|---|---|---|---|---|
| 1 | 4 | (50.0) | 2 | (66.7) |
| 2 | 1 | (12.5) | 0 | |
| Median number of | 2.5 | (1-4) | 2 | (2-3) |
| prior regimens, n (range) | | | | |
| Median time on | 4.2 | (1.4-5.8) | 2.8 | (1.5-2.9) |
| treatment, m (range) | | | | |
| Overall response rate^{a}, n (%) | 3 | (37.5) | 1^{b} | (33.3) |
| Clinical benefit rate^{c}, n (%) | 6 | (75.0) | 1^{b} | (33.3) |

| | | |
|---|---|---|
| Duration of PR, m | 0.7, 2.8, 2.8 | 0.1 |

| | | | | |
|---|---|---|---|---|
| Median PFS, m (95% CI) | 4.2 | (1.2-5.4) | 2.8 | (1.4-2.8) |

^{a}Complete response (CR) + PR.
^{b}Patient had an extracellular HER2 mutation (D277Y/D297Y).
^{c}CR + PR + SD for >4 months

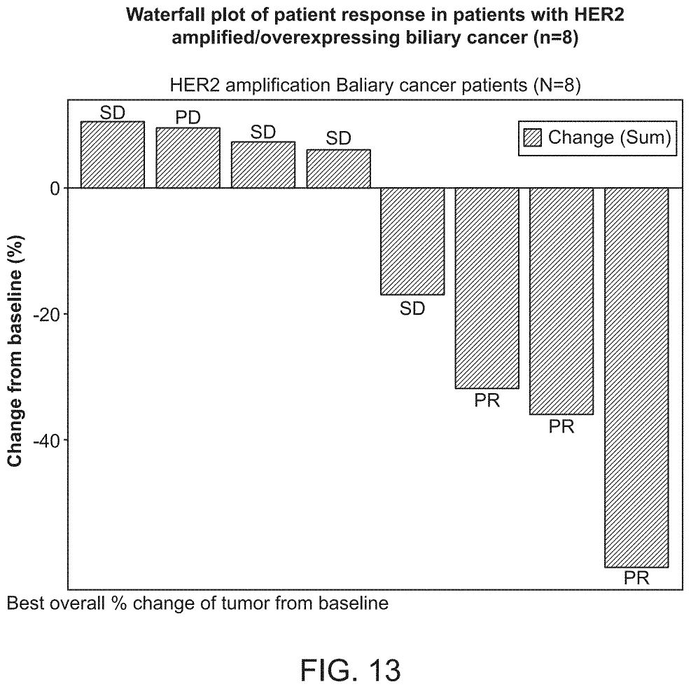

FIG. 13 shows a waterfall plot of treatment response in patients with HER2-amplified biliary cancer (N=8).

The results set forth in Table 6 above and shown in FIG. 13 indicate that pertuzumab+trastuzumab has activity in HER2 amplified/overexpressed/mutated metastatic biliary tumors, suggesting HER2 as a therapeutic target for these rare cancers.

### Example 4

### Pertuzumab+Trastuzumab for Treatment of Patients with HER2-Positive Metastatic Bladder Cancer (mBC)

Patients with mBC have few treatment options beyond the second-line setting. HER2 is amplified in 5-42% of BCs, but limited data are available regarding treatment with HER2-targeted agents.

12 patients with platinum-resistant HER2-positive mBC (HER2-amplified, n=9; HER2-mutated, n=3) enrolled in the MyPathway (NCR02091141) open-label, multicenter, phase IIA study described in Example 1 received standard doses of pertuzumab+trastuzumab. At a median follow-up of 5.4 (range 0.9-14.5) months, 1 patient had a complete response (CR, ongoing), 2 had partial responses (PR), and 2 had stable disease (SD) for >4 months (Table). Safety was consistent with the product labels. The results are summarized in Table 8.

TABLE 8

| | HER2- | | HER2- | |
|---|---|---|---|---|
| | amplified | | mutated | |
| | (n = 9)^{a} | | (n = 3) | |
| Median age, years (range) | 67 | (55-82) | 55 | (43-60) |
| Female | 2 | (22.2) | 1 | (33.3) |
| HER2 testing method, n (%)^{b} | | | | |
| Gene sequencing | 7 | (77.8) | 3 | (100.0) |
| FISH/CISH | 2 | (22.2) | N/A | |
| IHC | 2 | (22.2) | N/A | |
| ≥2 sites of metastases, n (%) | 8 | (88.9) | 3 | (100.0) |
| Median number of prior | 3 | (1-4) | 3 | (3-3) |
| regimens, n (range) | | | | |
| Median time on treatment, | 5.3 | (1.4-15.2) | 1.4 | (0.7-1.4) |
| months (range) | | | | |
| Overall response rate^{c}, n (%) | 3 | (33.3) | 0 | |
| Clinical benefit rate^{d}, n (%) | 5 | (55.6) | 0 | |
| Duration of CR/PR, months | 12.5 (CR), 5.5, 3.7 | | N/A | |
| Progressive disease, n (%) | 2 | (22.2) | 3 | (100.0) |

^{a}One patient also had mutated HER2.
^{b}Some patients had multiple tests.
^{c}CR + PR.
^{d}CR + PR + SD for >4 months.

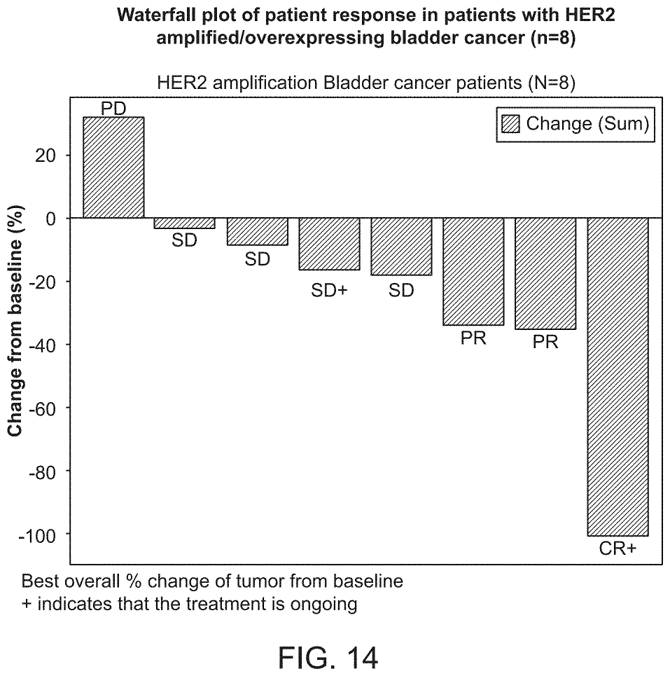

FIG. 14 shows a waterfall plot of treatment response in patients with HER2-amplified bladder cancer (N=8).

The results shown in Table 7 above and in FIG. 14 indicate that pertuzumab+trastuzumab has activity in HER2 amplified/overexpressed/mutated metastatic bladder tumors, suggesting HER2 as a therapeutic target for these rare cancers.

### Example 5

### Pertuzumab+Trastuzumab for Treatment of Patients with HER2-Positive Metastatic Urothelial Cancer (mUC)

Patients with metastatic urothelial cancer (mUC) have limited treatment options, primarily consisting of platinum-based chemotherapies as a first line treatment, and atezolizumab in the second line. There are no approved therapies beyond the second line. Accordingly, additional therapeutic options, particularly those with good tolerability, are needed.

Alterations in the HER2 receptor have been identified in patients with bladder and urothelial cancers, as shown in the following Table 9.

TABLE 9
Prevalence of HER2 alterations in patients with
bladder cancer/metastatic urothelial cancer (mUC)

| Database | Tissue | HER2 amplifications | | HER2 mutations | |
|---|---|---|---|---|---|
| COSMIC¹ | Urinary tract | 16/419 | (4%) | 59/1133 | (5%) |
| cBioPortal² | Bladder - urothelial | 6/99 | (6%) | 18/230 | (8%) |
| Foundation | Bladder - urothelial | 86/6535 | (1%) | 79/6535 | (1%) |
| Medicine³ | | | | | |
| Foundation Medicine | Bladder | 93/7582 | (1%) | 93/7582 | (1%) |

¹http://cancer.sanger.ac.uk/cosmic
²http://www.cbioportal.org

Methods

Patient Selection and Treatment

Patients in this subset analysis of the clinical trial described in Example 1 has metastatic urothelial cancer (mUC) having at least one of the following HER2 alterations:
HER2 amplification: next-generation sequencing (NGS) or fluorescent or chromogenic in situ hybridization (FISH or CISH; signal ration >2.0 or copy number >6)
HER2 overexpression: immunohistochemistry (IHC; 3+)
Potentially actionable HER2 mutations (i.e. insertions in exon 20, deletions around amino acids 755-759, known activating mutations, or mutations reported at least twice in the COSMIC database): NGS

Patients received pertuzumab (840 mg IV loading dose, followed by 420 mg IC every 3 weeks)+trastuzumab (8 mg/kg IV loading dose, followed by 6 mg/kg IV every 3 weeks) until disease progression or unacceptable toxicity. The primary endpoint was investigator-assessed objective response rate (ORR).

Assessment of Statistical Methods

Investigators carried out tumor assessments per RECIST (v1.1) (Eisenhauer E A, et al., Eur J Cancer, 2009; 45:228-247) every 6 weeks for the first 24 weeks and every 12 weeks thereafter. Confirmatory tumor evaluations were not required.

ORR is the percentage of patients with complete response (CR) or partial response (PR) at any time

Clinical benefit rate (CBR) is the percentage of patients with CR, PR, or stable disease (SD) for >4 months.

Duration of response is calculated from the date of first treatment response to the date of disease progression/death, whichever occurred earlier, or the date of the last tumor assessment for patients without disease progression/death.

Progression-free survival (PFS) was calculated as the time from the date of first treatment to the date if progression/death, or to the date of the last tumor assessment if there was no progressive disease/death.

Overall survival (OS) was calculated as the time from the date of first treatment to the date of death, or to the date last known to be alive if there was no death.

Results

Patients

By the cut-off date, 247 patients had been treated in the MyPathway study, including 12 patients with platinum-resistant HER2-positive mUC who received pertuzumab+trastuzumab treatment. Of these 12 patients, 9 exhibited HER2-amplification/overexpression, and 3 had putative HER2 activating mutations (S310Y, S310F, and deletion around amino acids 755-759). One patient in the HER2-amplified/overexpressed cohort also had a HER2 mutation (S310Y). Baseline demographics and clinical outcomes by patient are shown in Table 10.

Treatment Exposure and Clinical Outcomes

Median follow-up was 4.6 (range 1.0-16.6) months.

Among patients with HER2-amplification.overexpression (n=9):
Median time on treatment was 4.6 (range 0.7-16.6) months
ORR was 33.3% (95% confidence interval [CI] 7.5-70.1). Three patients responded to pertuzumab+trastuzumab, including one patient with an ongoing CR. The median duration of response was 5.5 (range 0.9-15.2) months. CBR was 55.6% (95% CI 21.2-86.3). Two patients had SD for >4 months.

Among patients with HER2-mutations (n=3):
Median time on treatment was 0.7 (range 0-0.8) months
No patients experienced an objective response or stable disease for >4 months

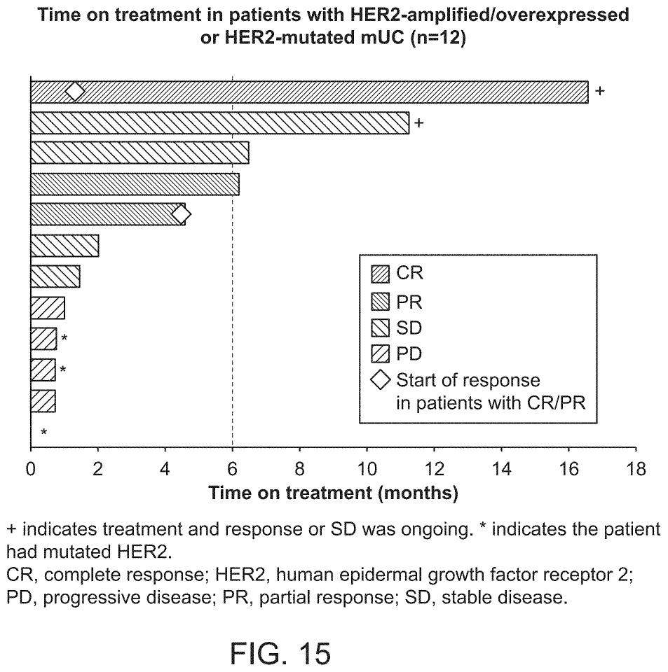

Time on treatment by patient is shown in FIG. 15.

The baseline characteristics and clinical outcomes of the individual patients are shown in Table 9.

TABLE 10
Baseline characteristics and clinical outcomes in patients
with HER2-amplified/overexpressed or HER2-mutated mUC

| | Baseline characteristics | | | | | |
|---|---|---|---|---|---|---|
| | | | Testing | HER2 | Prior lines | Sites of |
| Patient | Sex | Age | platform | alteration | of therapy | metastases |

HER2-amplified/overexpressed^{b}(n = 9)

| | | | | | | |
|---|---|---|---|---|---|---|
| 076 | M | 63 | NGS | Amplification | 2 | Peritoneum |
| 073 | M | 82 | FISH/CISH | Amplification | 3 | Lung, lymph |
| | | | | | | node |
| 007 | M | 67 | NGS, IHC | Overexpression + | 4 | Abdominal |
| | | | | amplification | | cavity, |
| | | | | | | lymph node |
| 136 | M | 61 | NGS | Amplification | 3 | Liver, lung, |
| | | | | | | lymph node |
| 060 | M | 77 | NGS | Amplification | 4 | Chest wall, |
| | | | | | | liver, lymph |
| | | | | | | node, skin |
| 081 | M | 67 | NGS | Amplification | 1 | Bone, brain, |
| | | | | | | liver, lymph |
| | | | | | | node |
| 142 | F | 62 | NGS | Amplification + | 2 | Bone, liver, |
| | | | | mutation^{c} | | lung, pelvic |
| | | | | | | sidewall |
| 098 | M | 72 | NGS | Amplification | 3 | Bone, lymph |
| | | | | | | node, pelvis, |
| | | | | | | peritoneum |
| 113 | F | 55 | FISH/CISH | Amplification | 2 | Liver, lung, |
| | | | | | | lymph node, |
| | | | | | | pelvic region |
| All HER2- | M: | Median: | — | — | Median: 3 | — |
| amplified/ | 77.8% | 67 | | | | |
| over- | F: | Range: | | | Range: 1-4 | |
| expressed | 22.2% | 55-82 | | | | |

HER2-mutated^{b} (n =3)

| | | | | | | |
|---|---|---|---|---|---|---|
| 125 | M | 60 | NGS | Deletion | 3 | Liver, lung, |
| | | | | (755-759) | | lymph node, |
| | | | | | | peritoneum, |
| | | | | | | seminal vesicle |
| 112 | F | 55 | NGS | S310F | 3 | Brain, liver, |
| | | | | | | lung, lymph |
| | | | | | | node |
| 038 | M | 43 | NGS | S310Y | 3 | Kidney, |
| | | | | | | liver, lung, |
| | | | | | | lymph node |
| All HER2- | M: 66.7% | Median: 55 | — | — | Median: 3 | — |
| mutated | | | | | | |
| All HER2- | F: 33.3% | Range: 43-60 | | | Range: 3-3 | |
| amplified/ | | | | | | |
| ALL | M: 75.0% | Median: 63 | — | — | Median: 3 | — |
| PATIENTS | F: 25.0% | Range: 43-82 | | | Range: 1-4 | |

| | Clinical outcomes | | | |
|---|---|---|---|---|
| | | CR/PR | | |
| | Best | duration, | PFS, | OS, |
| | response | months | months | months^{a} |
| HER2-amplified/overexpressed^{b}(n = 9) | | | | |
| 076 | CR+ | 15.2+ | 16.6+ | 16.6+ |
| 073 | PR | 5.5 | 6.9 | 12.5 |
| 007 | PR | 0.9 | 5.3 | 8.1 |
| 136 | SD+ | — | 11.0+ | 11.2+ |
| 060 | SD | — | 7.6 | 8.6 |
| 081 | SD | — | 3.3 | 3.3 |
| 142 | SD | — | 2.6 | 2.8 |
| 098 | PD | — | 1.7 | 2.3+ |
| 113 | PD | — | 1.3 | 1.4+ |
| All HER2- | ORR: 33.3% | Median | Median: 5.3 | Median: 8.6 |
| amplified/ | 95% CI: | CR/PR | 95% CI: | 95% CI: |
| over- | 7.5-70.1 | duration: | 1.3-NE | 2.8-NE |
| expressed | CBR: 55.6% | 5.5 | | |
| All HER2- | 95% CI: | Range: 0.9-NE | | |
| amplified/ | 21.2-86.3 | | | |
| HER2-mutated^{b} (n =3) | | | | |
| 125 | PD | — | 1.4 | 5.6 |
| 112 | PD | — | 1.3 | 3.7 |
| 038 | PD | — | 0.5 | 1.0 |
| All HER2- | ORR/CBR: | — | Median: 1.3 | Median: 3.7 |
| mutated | 0% | | 95% CI: | 95% CI: |
| | | | 0.5-1.4 | 1.0-5.6 |
| All HER2- | ORR: 25.0% | Median | Median: 2.9 | Median: 8.1 |
| amplified/ | 95% CI: | CR/PR | 95% CI: | 95% CI: |
| | 5.5-57.2 | duration: | 1.3-7.6 | 2.8-12.5 |
| ALL PATIENTS | CBR: 41.7% | 5.5 | | |
| | 95% CI: | Range: | | |
| | 15.2-72.3 | 0.9-NE | | |

+indicates measure was ongoing.
^{a}All patients who died did so due to disease progression.
^{b}Not all patients were tested for all alteration types.
^{c}S310Y.
CBR, clinical benefit rate; CI, confidence interval; CR, complete response; F, female; HER2, human epidermal growth factor receptor 2; M, male; NE, not estimable; ORR, objective response rate; PD, progressive disease; PR, partial response; SD, stable disease.

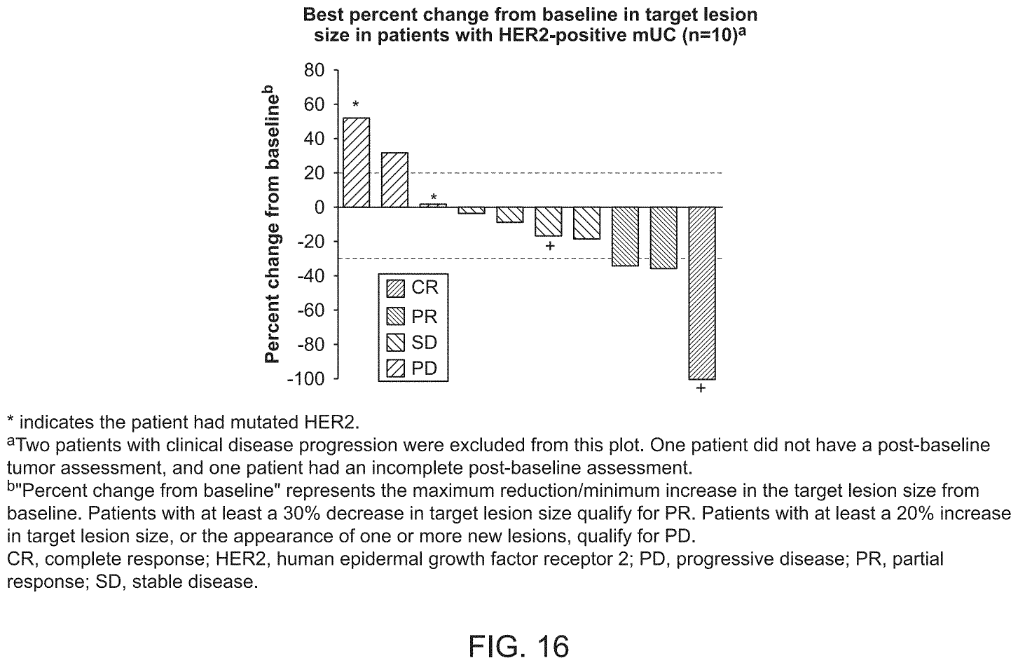

The best percent change from baseline in target lesion size by patient is shown in FIG. 16.

At the time of data cut-off, 77.8% (7/9) patients in the HERE2-amplification/overexpression cohort and all patients (3/3) in the HER2-mutated cohort had experienced an PFS event (disease progression [n=9] or death [n=1].

Median progression-free survival was 5.3 (95% CI, 1.3-NE) months in patients with HER2-amplification/overexpression and 1.3 (95% CI, 0.5-1.4) months in patients with HER2-mutated disease.

By data cut-off, 55.6% (5/9) of patients with HER2-amplification/overexpression and 100% (3/3) of patients with mutated HER2 had died, all due to disease progression. Median overall survival was 8.6 (95% CI, 1.8-NE) months in patients with HER2-amplification/overexpression and 3.7 (95% CI, 1.0-5.6) months in patients with HER2-mutated disease.

Case Study of a Patient with HER2-Amplified mUC

A 63-year-old while male patient presented with superficial bladder cancer in 2010 and was treated with multiple cycles of Bacillus Calmette-Guerin immunotherapy.

In late 2012, transurethral resection showed T1, grade 3 urothelial carcinoma. The patient underwent a left distal ureterectomy in January 2013; a resected lymph node was negative.

Nodal/soft tissue and osseous lesions were found throughout the body in August 2014, suggesting diffuse metastases. The patient received dose-dense treatment with methotrexate+vinblastine+doxorubicin+cisplatin for 7 cycles, with near-complete response.

Recurrent disease with peritoneal metastases was discovered in April 2015.

Gene sequencing identified HER2 amplification with a copy number of 52, upon which the patient enrolled in MyPathway and began treatment with pertuzumab+trastuzumab.

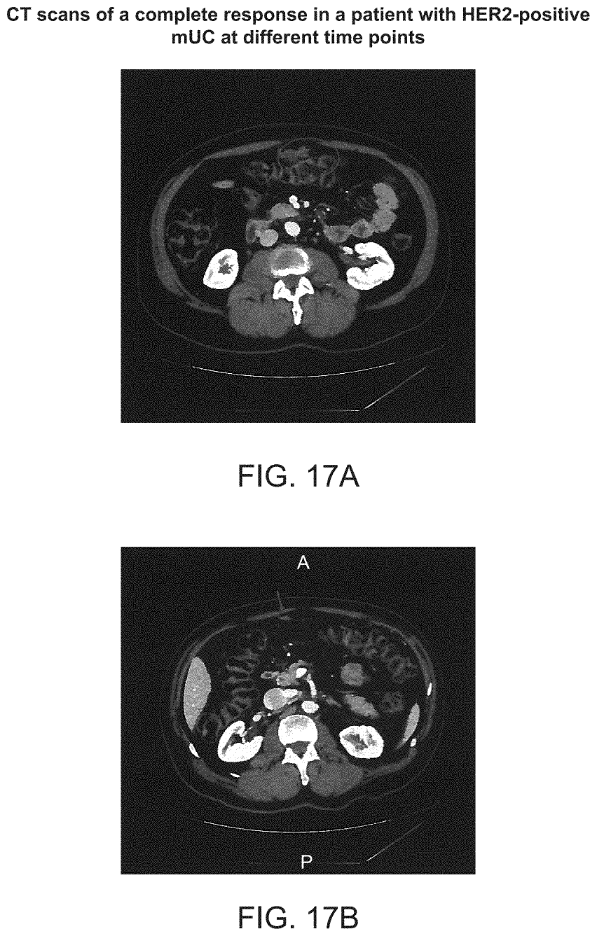

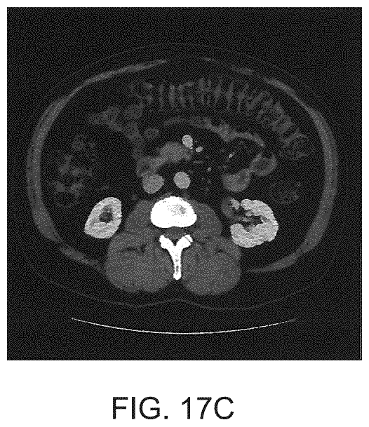

A response was observed after 2 cycles of therapy, continuing into a CR that was ongoing at the last tumor assessment (FIGS. 17A-C).

At data cut-off, the patient has received pertuzumab+trastuzumab for 16.6 months (25 cycles).

Safety

Safety was consistent with the product labels for pertuzumab and trastuzumab. Among all patients, 58.3% (n=7) experienced at least one treatment-related adverse event (AE) and 8.3% (n=1) experiences at least one treatment-related grade ≥3 AE.

### Conclusions

The disclosed results indicate that pertuzumab+trastuzumab may provide a well-tolerated treatment option for patients with previously treated HER2-amplified/overexpressed mUC.

In patients with HER2-amplified/overexpressed disease, ORR was 33.3% and CBR was 55.6%, where one patient with peritoneal metastases experienced a durable, ongoing CR (152+ months at the data cut-off), and three additional patients experienced PR or SD for greater than 6 months.

Although patient numbers were small, no activity was observed in patients with HER2-mutated mUC. The low rate of significant quality of life impairing toxicities observed with the chemotherapy-free targeted regimen of pertuzumab+trastuzumab may be particularly valuable in patients with mUC-related complications, such as low kidney function.

### Example 6

### Pertuzumab+Trastuzumab for Treatment of Patients with HER2-Positive Salivary Gland Carcinoma

Salivary gland cancers comprise <1% of cancers. Advanced cases have a 40% 5-year survival rate. Due to their rarity, no standard treatment guidelines exist. However, salivary duct carcinomas have morphological and gene expression profiles similar to breast cancers, and 20-40% of this subset have HER2 alterations.

Methods

Patients had advanced salivary cancer with HER2 (amplification, overexpression, and/or mutation), locally assessed by gene sequencing, FISH, or IHC, as applicable. Patients received standard doses of pertuzumab+trastuzumab, until disease progression or unacceptable toxicity, as described in Example 1. The primary endpoint was investigator-assessed objective response rate (ORR) by RECIST v1.1.

Results

At the data cut-off, 7 patients with HER2 alterations had been treated for salivary cancer, all carcinomas. One HER2 patient without a post-baseline tumor assessment by data cut-off was not evaluable for efficacy. Characteristics and outcomes are shown (Table 10). Of 6 patients with a complete response (CR) or partial response (PR), 5 patients were still receiving study treatment by the data cut off, with a median time on treatment of 4.6 months (range 1.4-12.5). There were no new safety signals.

TABLE 11

| | HER2 (n = 7)^{a} | | Hh (n = 1)^{b} |
|---|---|---|---|
| Median age, years | 59 | (range 47-80) | 65 |
| Male, n | 6 | (86%) | 1 |
| Prior lines of treatment, | 1 | (range 0-3) | 0 |
| median | | | |
| Objective | 5 | (1 CR, 4 PR) | 1 (PR) |
| response^{c,d}, n | ORR: 83% | (95% CI 36-100) | |

^{a}Six patients had HER2 amplification/overexpression (1 patient with PR also had HER2 mutations [D769H/L755F]). The unevaluable patient had a HER2 mutation (S310F).
^{b}PTCH-1 (Q400).
^{c}CR + PR.
^{d}In 6 evaluable patients.

### Conclusions

Of the patients with advanced salivary carcinoma characterized by HER2 alterations, 5 achieved CR or PR. These promising results merit study of these treatments in additional patients.

### Example 7

### Pertuzumab+Trastuzumab for Treatment of Patients with HER2-Positive Lung Cancer

Methods

Patients with previously treated advanced NSCLC and alterations in the HER2 (amplification and/or mutation), received standard doses of pertuzumab+trastuzumab as described in Example 1, until disease progression or unacceptable toxicity. The primary endpoint is investigator-assessed objective response rate (ORR, defined as complete response [CR]+partial response [PR]) by RECIST v1.1.

Results

The results are summarized in the Table 12.

TABLE 12

| | | | | Durations of |
|---|---|---|---|---|
| | | | Clinical | objective |
| | | | benefit | responses, |
| | Patients, n | ORR, n (%) | rate^{a}, n (%) | months |
| HER2 | 31 | 6 (19%) | 10 (32%) | <1+, 3+, 5+, |
| alterations^{b} | | | | 6, 8, 10 |

+indicates response is ongoing.
^{a}CR + PR + stable disease >4 months.
^{b}HER2 amplified and/or mutated.

### CONCLUSIONS

Targeted therapy is active in patients with previously treated NSCLC harboring HER2 alterations (amplifications and/or mutations).

Overall, the results presented in the Examples confirm that efficacy of pertuzumab+trastuzumab targeted therapy to treat in a number of advanced, metastatic, difficult to treated cancers.

While certain embodiments of the present invention have been shown and described herein, it will be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for the treatment of advanced colorectal cancer, comprising administering to a human patient with HER2-positive, HER2-amplified, or HER2-mutated advanced colorectal cancer an effective amount of a combination of pertuzumab and trastuzumab.

2. A method for the treatment of advanced biliary cancer, comprising administering to a human patient with HER2-positive, HER2-amplified, or HER2-mutated advanced biliary cancer an effective amount of a combination of pertuzumab and trastuzumab.

3. A method for the treatment of advanced urothelial cancer, comprising administering to a human patient with HER2-positive, HER2-amplified, or HER2-mutated advanced bladder cancer an effective amount of a combination of pertuzumab and trastuzumab.

4. A method for the treatment of advanced bladder cancer, comprising administering to a human patient with HER2-positive, HER2-amplified, or HER2-mutated advanced bladder cancer an effective amount of a combination of pertuzumab and trastuzumab.

5. A method for the treatment of advanced salivary cancer, comprising administering to a human patient with HER2-positive, HER2-amplified, or HER2-mutated advanced bladder cancer an effective amount of a combination of pertuzumab and trastuzumab.

6. A method for the treatment of advanced lung cancer, comprising administering to a human patient with HER2-positive, HER2-amplified, or HER2-mutated advanced bladder cancer an effective amount of a combination of pertuzumab and trastuzumab.

7. A method for the treatment of advanced pancreatic cancer, comprising administering to a human patient with HER2-positive, HER2-amplified, or HER2-mutated advanced pancreatic cancer an effective amount of a combination of pertuzumab and trastuzumab.

8. A method for the treatment of advanced ovarian cancer, comprising administering to a human patient with HER2-positive, HER2-amplified, or HER2-mutated advanced ovarian cancer an effective amount of a combination of pertuzumab and trastuzumab.

9. A method for the treatment of advanced prostate cancer, comprising administering to a human patient with HER2-positive, HER2-amplified, or HER2-mutated advanced prostate cancer an effective amount of a combination of pertuzumab and trastuzumab.

10. A method for the treatment of advanced skin cancer, comprising administering to a human patient with HER2-positive, HER2-amplified, or HER2-mutated advanced skin cancer an effective amount of a combination of pertuzumab and trastuzumab.

11. The method of any one of claims 1-10, wherein the cancer is HER2-positive.

12. The method of claim 11, wherein the HER2 expression level is IHC 2+ or 3+.

13. The method of any one of claims 1-10, wherein the cancer is HER2-amplified.

14. The method of claim 13, wherein HER2 amplification is determined by fluorescence in situ hybridization (FISH).

15. The method of claim 13, wherein HER2 amplification is determined by next generation sequencing (NGS).

16. The method of any one of claims 1-10, wherein the cancer is HER2-mutated.

17. The method of claim 16, wherein the mutation is a HER2-activating mutation.

18. The method of claim 16, wherein the HER2 mutation is selected from the group consisting of insertions within exon 20 of HER2, deletions around amino acid residues 755-759 of HER2, G309A, G309E, S310F, D769H, D769Y, V777L, P780-Y781insGSP, V8421I, R896C and other putative activating mutations found two or more unique specimens.

19. The method of any one of claims 1-10, wherein the cancer is locally advanced.

20. The method of any one of claims 1-10, wherein the cancer is metastatic.

21. The method of any of claims 1-20, wherein the cancer is refractory to another treatment regimen.

22. The method of claim 21, wherein the cancer is chemotherapy-resistant.

23. The method of claim 22, wherein the cancer is platinum-resistant.

24. The method of any one of claims 1-23, wherein the patient received one to five rounds of prior treatments for said cancer.

25. The method of claim 24, wherein said prior treatments comprise chemotherapy.

26. The method of claim 24 or 25, wherein said prior treatments comprise HER2-directed therapy.

27. The method of claim 24, wherein at least one of said prior treatments administered in the advanced stage.

28. The method of claim 24, wherein at least one of said prior treatment is neoadjuvant treatment.

29. The method of claim 24, wherein at least one of said prior treatments is adjuvant treatment.

30. The method of claim 24, wherein said cancer is resistant to at least one of said prior treatments.

31. The method of any one of claims 1-30, wherein the combination of pertuzumab and trastuzumab is administered in the absence of other anti-cancer drug(s).

32. The method of claim 31, wherein the combination of pertuzumab and trastuzumab is administered in the absence of chemotherapy.

33. The method of claim 31, wherein the combination of pertuzumab and trastuzumab is administered in the absence of another HER2 directed therapy.

34. The method of any one of claims 1-33, wherein said treatment consists essentially of combined administration of a combination of pertuzumab and trastuzumab

35. The method of any one of claims 1-34, wherein said administration results in improved overall response rate (ORR) relative to administration of pertuzumab or trastuzumab as a single agent.

36. The method of any one of claims 1-35, wherein said administration results in improved partial response (PR) relative to administration of pertuzumab or trastuzumab as a single agent.

37. The method of any one of claims 1-35, wherein said administration results in improved complete response (CR) relative to administration of pertuzumab or trastuzumab as a single agent.

38. The method of any one of claims 1-35, wherein said administration extends survival of said patient relative to administration of pertuzumab or trastuzumab as a single agent.

39. The method of claim 38, wherein said administration extends progression-free survival (PFS).

40. The method of claim 38, wherein said administration extends overall survival (OS).

41. The method of any one of claims 1-35, wherein the combination of pertuzumab and trastuzumab results in a synergistic effect.

42. The method of any one of claims 1-41, wherein said administration does not result in an increase of side-effects relative to monotherapy with pertuzumab or trastuzumab.

43. The method of claim 42, wherein said administration does not result in an increase of cardiac-side-effects relative to monotherapy with pertuzumab or trastuzumab.

44. An article of manufacture comprising a vial with pertuzumab and a package insert, wherein the package insert provides instructions to administer said pertuzumab as claimed in any one of claims 1 to 43.

45. A composition of pertuzumab for use, in combination with trastuzumab, for the treatment of a human patient with HER2-positive, HER2-amplified, or HER2-mutated advanced colorectal cancer.

46. A composition of pertuzumab for use, in combination with trastuzumab, for the treatment of a human patient with HER2-positive, HER2-amplified, or HER2-mutated advanced biliary cancer.

47. A composition of pertuzumab for use, in combination with trastuzumab, for the treatment of a human patient with HER2-positive, HER2-amplified, or HER2-mutated advanced urothelial cancer.

48. A composition of pertuzumab for use, in combination with trastuzumab, for the treatment of a human patient with HER2-positive, HER2-amplified, or HER2-mutated advanced bladder cancer.

49. A composition of pertuzumab for use, in combination with trastuzumab, for the treatment of a human patient with HER2-positive, HER2-amplified, or HER2-mutated advanced salivary cancer.

50. A composition of pertuzumab for use, in combination with trastuzumab, for the treatment of a human patient with HER2-positive, HER2-amplified, or HER2-mutated advanced lung cancer.

51. A composition of pertuzumab for use, in combination with trastuzumab, for the treatment of a human patient with HER2-positive, HER2-amplified, or HER2-mutated advanced pancreatic cancer.

52. A composition of pertuzumab for use, in combination with trastuzumab, for the treatment of a human patient with HER2-positive, HER2-amplified, or HER2-mutated advanced ovarian cancer.

53. A composition of pertuzumab for use, in combination with trastuzumab, for the treatment of a human patient with HER2-positive, HER2-amplified, or HER2-mutated advanced prostate cancer.

54. A composition of pertuzumab for use, in combination with trastuzumab, for the treatment of a human patient with HER2-positive, HER2-amplified, or HER2-mutated advanced skin cancer.

55. Use of pertuzumab in the preparation of a medicament for the treatment of a human patient with HER2-positive, HER2-amplified, or HER2-mutated advanced colorectal cancer, in combination with trastuzumab.

56. Use of pertuzumab in the preparation of a medicament for the treatment of a human patient with HER2-positive, HER2-amplified, or HER2-mutated advanced biliary cancer, in combination with trastuzumab.

57. Use of pertuzumab in the preparation of a medicament for the treatment of a human patient with HER2-positive, HER2-amplified, or HER2-mutated advanced urothelial cancer, in combination with trastuzumab.

58. Use of pertuzumab in the preparation of a medicament for the treatment of a human patient with HER2-positive, HER2-amplified, or HER2-mutated advanced bladder cancer, in combination with trastuzumab.

59. Use of pertuzumab in the preparation of a medicament for the treatment of a human patient with HER2-positive, HER2-amplified, or HER2-mutated advanced salivary cancer, in combination with trastuzumab.

60. Use of pertuzumab in the preparation of a medicament for the treatment of a human patient with HER2-positive, HER2-amplified, or HER2-mutated advanced lung cancer, in combination with trastuzumab.

61. Use of pertuzumab in the preparation of a medicament for the treatment of a human patient with HER2-positive, HER2-amplified, or HER2-mutated advanced pancreatic cancer, in combination with trastuzumab.

62. Use of pertuzumab in the preparation of a medicament for the treatment of a human patient with HER2-positive, HER2-amplified, or HER2-mutated advanced ovarian cancer, in combination with trastuzumab.

63. Use of pertuzumab in the preparation of a medicament for the treatment of a human patient with HER2-positive, HER2-amplified, or HER2-mutated advanced prostate cancer, in combination with trastuzumab.

64. Use of pertuzumab in the preparation of a medicament for the treatment of a human patient with HER2-positive, HER2-amplified, or HER2-mutated advanced skin cancer, in combination with trastuzumab.
